(19) United States
(12) Patent Application Publication
(10) Pub. No.: US 2007/0244162 A1
(43) Pub. Date: Oct. 18, 2007

(54) SUBSTITUTED AROMATIC HETEROCYCLIC COMPOUNDS AS FUNGICIDES

(76) Inventors: Shy-Fuh Lee, Sunnyvale, CA (US); Micah Gliedt, Sunnyvale, CA (US); Richard Anderson, Palo Alto, CA (US)

Correspondence Address: MYERS BIGEL SIBLEY & SAJOVEC, PO BOX 37428, RALEIGH, NC, 27627, US

(21) Appl. No.: 11/611,398

(22) Filed: Dec. 15, 2006

Related U.S. Application Data
(60) Provisional application No. 60/751,558, filed on Dec. 19, 2005.

(51) Int. Cl. A61K 31/44

(52) U.S. Cl. 514/336; 514/340; 546/276.4; 546/280.4; 546/283.4

(57) ABSTRACT

The present invention provides compounds of formula I: 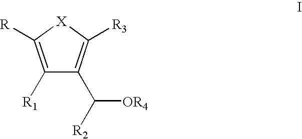
wherein X is S, O, or NR₅, along with salts thereof and compositions containing the same. The compounds are useful as, among other things, crop protection agents to combat or prevent fungal infestations, or to control other pests such as weeds, insects, or acarids that are harmful to crops.

### RELATED APPLICATIONS

This application claims the benefit of U.S. provisional patent application Ser. No. 60/751,558, filed Dec. 19, 2005, the disclosure of which is incorporated by reference herein in its entirety.

### FIELD OF THE INVENTION

The present invention concerns substituted aromatic heterocyclic compositions such as thiophenes, furans and pyrroles, and methods of use thereof for the control of microbial pests, particularly fungal pests, on plants.

### BACKGROUND OF THE INVENTION

The incidence of serious fungal infections, either systemic or topical, continues to increase for plants, animals, and humans. Many fungi are common in the environment and not harmful to plants or mammals. However, some fungi can produce disease in plants, humans and/or animals.

Fungicides are compounds, of natural or synthetic origin, which act to protect plants against damage caused by fungi, including oomycetes. Current methods of agriculture rely heavily on the use of fungicides. In fact, some crops cannot be grown usefully without the use of fungicides. Using fungicides allows a grower to increase the yield of the crop and consequently, increase the value of the crop. Numerous fungicidal agents have been developed. However, the treatment of fungal infestations and infections continues to be a major problem. Furthermore, fungicide and antifungal drug resistance has become a serious problem, rendering these agents ineffective for some agricultural and therapeutic uses. As such, a need exists for the development of new fungicidal and antifungal compounds (see, e.g., U.S. Pat. No. 6,673,827; See also U.S. Pat. No. 6,617,330 to Walter, which describes pyrimidin-4-enamine as fungicides).

### SUMMARY OF THE INVENTION

A first aspect of the invention is compounds of formula I: 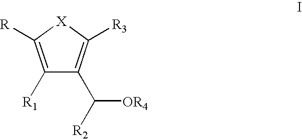
wherein:

X is S, O, or NR₅;

R is H; alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₁ is alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₂ is alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; heteroaryl, especially 2-, 3- or 4-pyridyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; 5-pyrimidinyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or 2- or 5-thiazolyl optionally substituted (e.g. 1, 2, 3 or 4 times) with halogen, alkyl, alkenyl, alkynyl, alkoxy, alkylthio, haloalkyl, haloalkenyl, haloalkoxy, haloalkylthio, cyano, or nitro;

R₃ is alkyl; alkoxyalkyl; haloalkyl; arylalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₄ is H; acyl (e.g., acetyl, benzoyl, phenylacetyl); haloacyl; alkoxycarbonyl; aryloxycarbonyl; alkylaminocarbonyl; or dialkylaminocarbonyl;

R₅ is H; alkyl; alkenyl; alkynyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

and salts thereof.

The present invention also concerns compositions comprising or consisting essentially of an active compound as described herein in combination with a suitable carrier (e.g., an agricultural carrier).

The compounds and compositions of the present invention are useful as crop protection agents to combat or prevent fungal infestations, or to control other pests such as weeds, insects, or acarids that are harmful to crops.

A second aspect of the present invention is a composition for controlling and preventing plant pathogenic microorganisms comprising, in combination, an active compound as described herein together with a suitable carrier.

A third aspect of the present invention is a method of controlling or preventing infestation of cultivated plants by pathogenic microorganisms, comprising applying an active compound as described herein to said plants, parts thereof or the locus thereof in an amount effective to control said microorganisms.

A further aspect of the present invention is a method of controlling or preventing infestation of technical materials by pathogenic microorganisms, comprising applying an active compound as described herein to said technical materials, parts thereof or the locus thereof in an amount effective to control said microorganisms.

A further aspect of the present invention is a method of treating a fungal infection in a subject in need thereof, comprising administering an active compound as described herein to said subject in an amount effective to treat said fungal infection.

A still further aspect of the present invention is the use of an active compound as described herein for the preparation of a composition (e.g., an agricultural formulation, a pharmaceutical formulation) for carrying out a method as described herein (e.g., an agricultural treatment as described herein, the treatment of technical materials as described herein, the treatment of a fungal infection in a subject as described herein).

The foregoing and other objects and aspects of the present invention are explained in greater detail below.

### DETAILED DESCRIPTION OF THE PREFERRED EMBODIMENTS

“Alkyl” as used herein refers to a saturated hydrocarbon radical which may be straight-chain or branched-chain (for example, ethyl, isopropyl, t-amyl, or 2,5-dimethylhexyl) or cyclic (for example cyclobutyl, cyclopropyl or cyclopentyl) and contains from 1 to 24 carbon atoms. This definition applies both when the term is used alone and when it is used as part of a compound term, such as “haloalkyl” and similar terms. In some embodiments, preferred alkyl groups are those containing 1 to 4 carbon atoms, which are also referred to as “lower alkyl.” In some embodiments preferred alkyl groups are those containing 5 or 6 to 24 carbon atoms, which may also be referred to as “higher alkyl”.

“Alkenyl,” as used herein, refers to a straight or branched chain hydrocarbon containing from 2 to 24 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 1 to 4 carbon atoms.

“Alkynyl,” as used herein, refers to a straight or branched chain hydrocarbon group containing from 2 to 24 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkyl and refers to a straight or branched chain hydrocarbon group containing from 1 to 4 carbon atoms.

“Alkoxy” refers to an alkyl radical as described above which also bears an oxygen substituent which is capable of covalent attachment to another hydrocarbon radical (such as, for example, methoxy, ethoxy and t-butoxy).

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Aryl” or “aromatic ring moiety” refers to an aromatic substituent which may be a single ring or multiple rings which are fused together, linked covalently or linked to a common group such as an ethylene or methylene moiety. The aromatic rings may each contain heteroatoms and hence “aryl” encompasses “heteroaryl” as used herein. Representative examples of aryl include, azulenyl, indanyl, indenyl, naphthyl, phenyl, tetrahydronaphthyl, biphenyl, diphenylmethyl, 2,2-diphenyl-1-ethyl, thienyl, pyridyl and quinoxalyl. “Aryl” means substituted or unsubstituted aryl unless otherwise indicated and hence the aryl moieties may be optionally substituted with halogen atoms, or other groups such as nitro, carboxyl, alkoxy, phenoxy and the like. Additionally, the aryl radicals may be attached to other moieties at any position on the aryl radical which would otherwise be occupied by a hydrogen atom (such as, for example, 2-pyridyl, 3-pyridyl and 4-pyridyl).

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom S may be substituted with one or two oxo groups, which may be shown as ═O.

“Agriculturally acceptable salt” means a salt the cation of which is known and accepted in the art for the formation of salts for agricultural or horticultural use. Preferably the salts are water-soluble.

“Cyano” as used herein refers to a —CN group.

“Halo” or “halogen,” as used herein, refers to —Cl, —Br, —I or —F.

“Haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Hydroxy,” as used herein, refers to an —OH group.

“Nitro,” as used herein, refers to a —NO₂ group.

“Oxy,” as used herein, refers to a —O— moiety.

“Thio,” as used herein, refers to a —S— moiety.

The disclosures of all U.S. Patent references cited herein are to be incorporated herein in their entirety as if fully set forth.

2. Compounds. The compounds of this invention are represented by formula I, including formulas Ia-Ic: 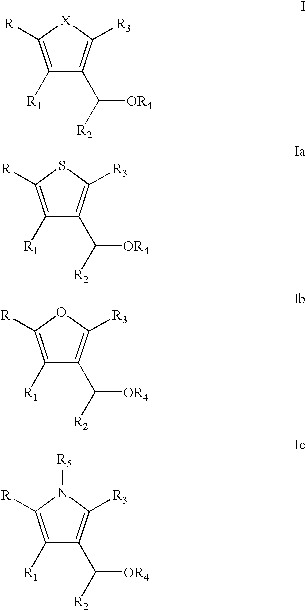
wherein:

X is S, O, or NR₅;

R is H; alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₁ is alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₂ is alkyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; heteroaryl, especially 2-, 3- or 4-pyridyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; 5-pyrimidinyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or 2- or 5-thiazolyl optionally substituted (e.g. 1, 2, 3 or 4 times) with halogen, alkyl, alkenyl, alkynyl, alkoxy, alkylthio, haloalkyl, haloalkenyl, haloalkoxy, haloalkylthio, cyano, or nitro;

R₃ is alkyl; alkoxyalkyl; haloalkyl; arylalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl;

R₄ is H; acyl (e.g., acetyl, benzoyl, phenylacetyl); haloacyl; alkoxycarbonyl; aryloxycarbonyl; alkylaminocarbonyl; or dialkylaminocarbonyl;

R₅ is H; alkyl; alkenyl; alkynyl; alkoxyalkyl; haloalkyl; arylalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryloxyalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; arylthioalkyl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g., 1, 2, 3 or 4 times) with halogen, alkyl, alkenyl, alkynyl, haloalkyl, haloalkenyl, alkoxy, alkylthio, haloalkoxy, haloalkylthio, cyano, or nitro; or alkylsilyl.

Methods of making. Compositions of generic structure Ia wherein R and R₄=H may be prepared by the [3+2]-cycloaddition of an acetylenethiolate anion II and an acetylenic ketone III to give thiophene ketone IV which upon reduction provides the corresponding thiophene alcohol Ia (see L. S. Rodinova, M. L. Petrov, and A. A. Petrov, Zhurnal Organicheskoi Khimii 1981, 17(10), 2071-2075 for a related thiophene synthesis): 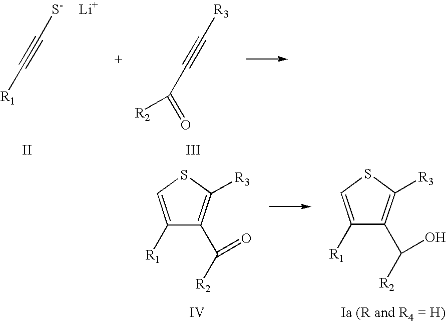
The [3+2]-cycloaddition reaction is carried out by preforming the acetylenethiolate in an inert solvent such as THF (tetrahydrofuran) at low temperature, preferably −78°, and then adding it to a solution of the acetylenic ketone III in an inert solvent or solvent mixture, such as THF and acetonitrile, at temperatures ranging from 0° C. to −20° C. The acetylenethiolate II is prepared from the reaction of sulfur with a lithium salt (VI) of a termninal acetylene V (H. G. Raubenheimer, G. J. Kruger, C. F. Marais, R. Otte, and J. T. Z. Hattingh, Organometallics 1988, 7, 1853-1858):

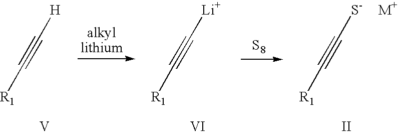

Lithium acetylide VI is formed by the treatment of terminal acetylene V with a strong base such as n-butyllithium in an inert solvent such as THF at low temperature, preferably from −40° C. to −78° C. Addition of sulfur to acetylide VI at low temperature (−40° C. to −78° C.) and reaction for 1.5-3 hr gives the acetylenethiolate II. Reduction of thiophene ketone IV is effected with a reducing agent such as LiAlH₄ in an inert solvent such as ether or THF, or NaBH₄ in a solvent such as ethanol at temperatures in the range of 0° C. to 20° C.

Alternatively, the Heck reaction may be employed to arylate activated thiophenes that are intermediates in the synthesis of Ia when R₁ and R₃ are aryl (L. Lavenot, C. Gozzi, K. Ilg, I Orlova, V. Penalva, and M. Lemaire, Journal of Organometallic Chem. 1998, 567, 49-55). Thus, thiophene-3-carboxaldehyde VII may be selectively arylated with aryl iodide R₃I in the presence of a transition metal catalyst such as a palladium(II) catalyst to give a 2-arylated intermediate VIII. A second palladium-catalyzed arylation with another aryl iodode R₁I then gives the 2,4-diarylthiophen-3-carboxaldehyde IX. Treatment of IX with 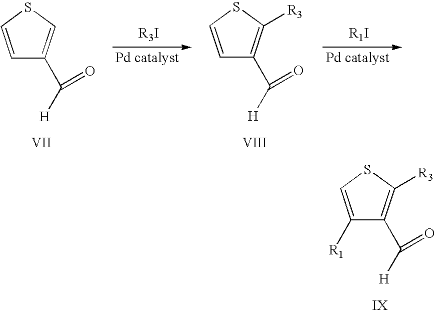
an organometallic reagent R₂M produces the compositions of generic structure Ia (R and R₄=H).

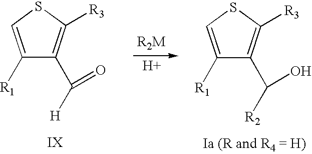

The Heck reaction is typically carried out in solvents such as acetonitrile or water, or in mixtures of the two, at temperatures in the range of 20-80° C. for 4-72 hrs. The typical palladium catalysts are palladium chloride, usually used in association with lithium chloride, or palladium acetate used with tetra-n-butylammonium bromide with or without a phosphine such as triphenylphosphine.

The addition of the organometallic reagent R₂M is typically conducted in an inert solvent such as ether or THF under N₂ atmosphere at 0-20° C. for 1-5 hrs. The organometallic reagent may be an organolithium reagent, or preferably an organomagnesium reagent.

Compositions of generic structure Ia wherein R₄=H may also be prepared by the Michael addition of a substituted α-mercaptoketone X (R′=H) to acetylenic ketone III to give the dihydrothienyl intermediate XI. Dehydration of XI to thiophene XII and subsequent 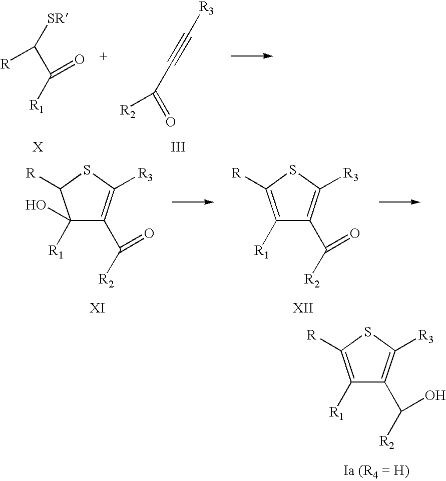
reduction of XII provides the composition Ia (R₄=H).

The Michael addition is carried out by reaction of the a-mercaptoketone X (R′=H) and the acetylenic ketone III in the presence of a base, preferably an organic base such as morpholine, and an inert solvent such as diethoxymethane at elevated temperatures such as reflux temperature for 1-8 hrs. Alternatively, α-acetylthioketone X (R′=COCH₃) may be used in the Michael addition wherein the base such as morpholine cleaves the thioester to the requisite α-mercaptoketone X (R′=H) in situ.

Intermediate XI is efficiently dehydrated by treatment with p-toluenesulfonic acid or acetic anhydride in toluene at elevated temperatures (80-100° C.) for 12-48 hrs. to produce the thienyl ketone XII, reduction of which is accomplished as above with a reducing agent such as LiAlH₄ in an inert solvent such as ether or THF, or NaBH₄ in a solvent such as ethanol at temperatures in the range of 0° C. to 20° C.

The α-acetylthioketones X (R′=COCH₃) and α-mercaptoketones X (R′=H) are readily 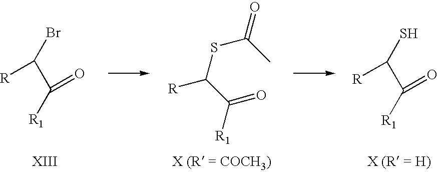
available by treating the corresponding α-bromoketones XIII with thioacetic acid in a basic medium to give X (R′=COCH₃), which upon treatment with aqueous base (e.g., aqueous NaOH) produces X (R′=H).

The compositions Ib may be prepared from XIII (R=H) or its chloro analog by reaction with the β-ketoester XIV under base catalyzed conditions to give the dihydrofuran XV (see F. Feist, Chem, Ber. 1902, 35, 1537-44), dehydration of which produces the furan XVI. This dehydration is efficiently effected by treatment of XV with p-toluenesulfonic acid or acetic anhydride in toluene at elevated temperatures (80-100° C.) for 12-48 hrs. Reduction of furyl ester XVI to furyl alcohol XVII and subsequent oxidation to furylcarboxaldehyde XVIII followed by addition of organometallic reagent R₂Li or R₂MgX′ gives compound Ib (R₄=H) The reduction of XVI to alcohol XVII is accomplished using a hydride reagent such as LiAlH₄ or diisobutylaluminum hydride (DIBAL) in an inert solvent such as ether of THF. Oxidation of XVII to aldehyde my be effected with reagents including activated MnO₂, o-iodosobenzoic acid (IBX) in DMSO, or CrO₃/pyr in inert solvents such as dichloromethane. The addition of the organometallic reagent to aldehyde XVIII is typically conducted in an inert solvent such as ether or THF under N₂ atmosphere at 0-20° C. for 1-5 hrs. The organometallic reagent may be an organolithium reagent, or preferably an organomagnesium reagent.

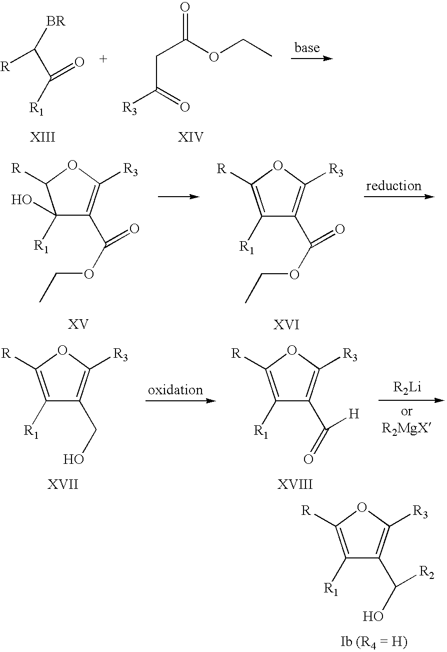

Alternatively, furyl ester XVI may be hydrolyzed to furoic acid XIX under aqueous basic conditions such as aqueous NaOH or LiOH. Conversion of the acid XIX to the Weinreb amide XX may be accomplished by coupling XIX and N,O-hydroxylamine hydrochloride using 1-hydroxybenzotriazole (HOBT) and diisopropylcarbodiimide (DIC) in the presence of diisopropylethylamine (DIEA) in an inert solvent such as dichloromethane (DCM). Addition of organometallic agent R₂MgX′ to XX in an inert solvent such as ether or THF under N₂ atmosphere at 0-20° C. for 1-5 hrs gives the ketone XXI, reduction of which is accomplished as above with a reducing agent such as LiAlH₄ in an inert solvent such as ether or THF, or NaBH₄ in a solvent such as ethanol at temperatures in the range of 0° C. to 20° C. to produce compound Ib (R₄=H).

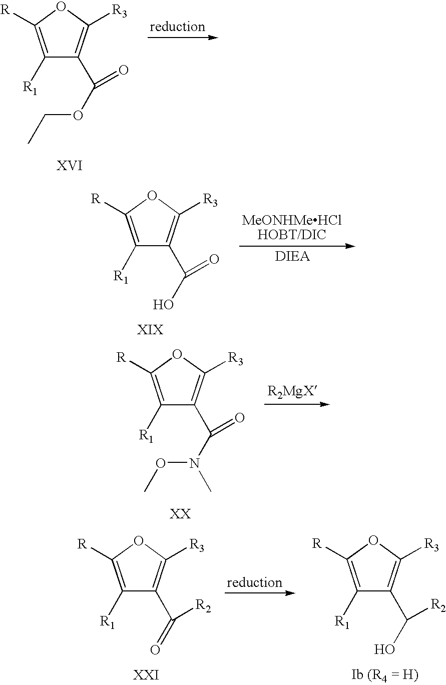

The compositions Ic may be prepared using an approach similar to that employed for the thiophenes Ia, i.e., but adding α-aminoketones XXII to the alkynylketone III in the Michael addition. Dehydration of dihydropyrrole XXIII to yield pyrrolyl ketone XXIV and subsequent reduction with LiAlH₄ or NaBH₄ gives Ic (R₄=H). Reaction conditions similar to those used to prepare the aforementioned furans Ib may be employed.

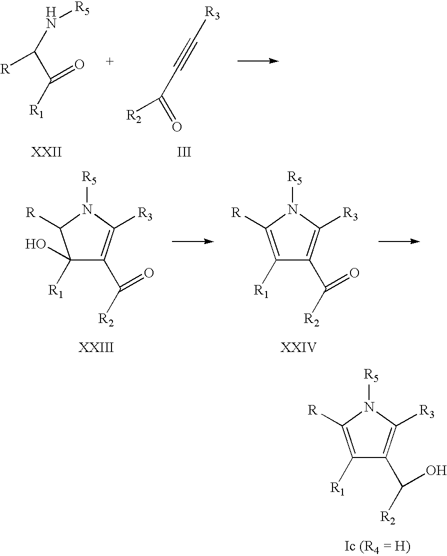

Alternatively, condensation of α-aminoketone XXII and , βketoester XIV under basic conditions gives the dihydropyrrole XXV (see L. Knorr, Chem. Ber. 1884, 17, 1635; A. H. Corwin, Heterocyclic Compounds, 1950, 1, 287), dehydration of which produces the pyrrolyl ester XXVI. Alkylation of XXVI with R₅I yields the N-substituted pyrrolyl ester XXVVII. In reactions similar to those described for the furan system, ester XXVII is converted to compound Ic (R₄=H).

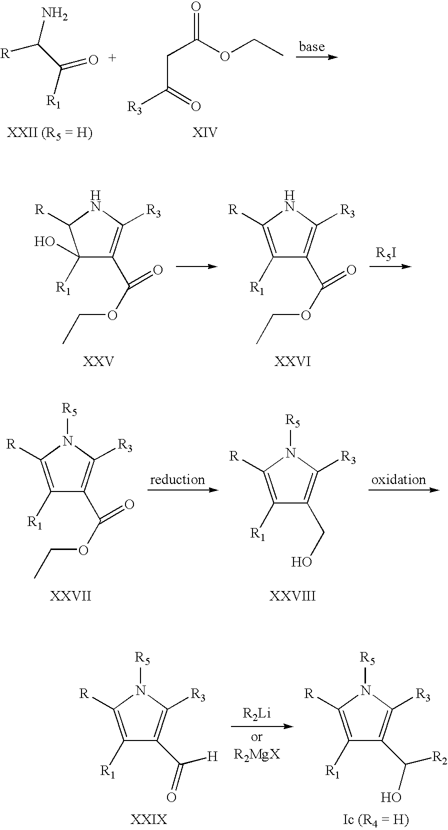

Ester XXVII may also be hydrolyzed to its corresponding acid XXX and converted to its Weinreb amide XXXI as above. Addition of organometallic agent R₂MgX′ gives the ketone XXXII, reduction of which produces compound Ic (R₄=H).

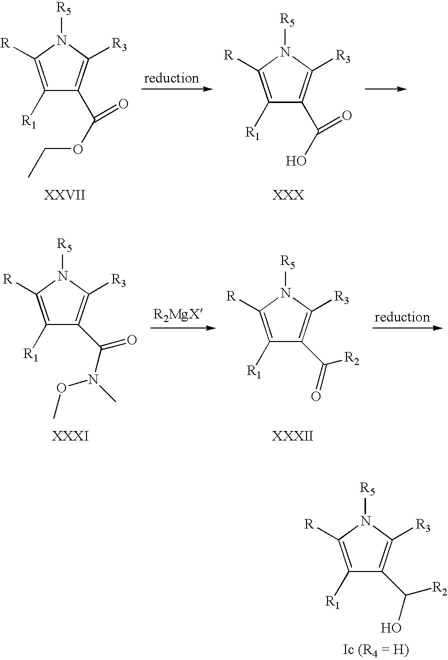

Exemplary compounds. Compounds Ia of the invention that are especially useful for the control of fungal pathogens are those in which:

R=H or alkyl;

R₁=aryl optionally substituted (e.g. 1, 2, 3 or 4 times) with halogen, alkyl, alkenyl, alkynyl, haloalkyl, alkoxy, alkylthio, haloalkoxy, cyano, nitro; or heteroaryl optionally substituted (e.g. 1, 2, 3 or 4 times) with halogen, alkyl, alkenyl, alkynyl, haloalkyl, alkoxy, alkylthio, haloalkoxy, cyano, nitro;

R₂=heteroaryl, especially 2-, 3- or 4-pyridyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, nitro; or 5-pyrimidinyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro;

R₃=alkyl; aryloxyalkyl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, or nitro; aryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, nitro; heteroaryl optionally substituted (e.g. 1, 2, 3 or 4 times) with halogen, alkyl, alkenyl, alkynyl, haloalkyl, haloalkenyl, alkoxy, alkylthio, haloalkoxy, haloalkylthio, cyano, nitro; or alkylsilyl;

R₄=H; and

R₅ is H, alkyl, or haloalkyl.

Examples of compounds of the present invention include, but are not limited to, the following:

| Compound | | |
|---|---|---|
| No. | Structure | Chemical Name |
| 1 | 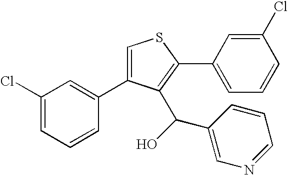 | 2,4-Bis-(3-chlorophenyl)-3-[(3- pyridyl)hydroxymethyl]- thiophene |
| 2 | 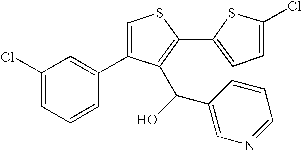 | 4-(3-Chlorophenyl)-2-(5- chloro-2-thienyl)-3-[(3- pyridyl)hydroxymethyl]- thiophene |
| 3 | 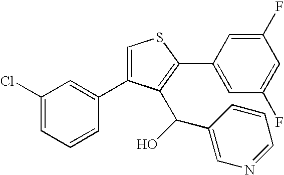 | 4-(3-Chlorophenyl)-2-(3,5- difluorophenyl)-3-[(3- pyridyl)hydroxymethyl]- thiophene |
| 4 | 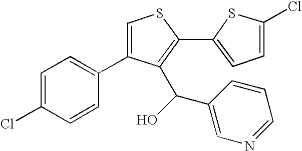 | 4-(4-Chlorophenyl)-2-(5- chloro-2-thienyl)-3-[(3- pyridyl)hydroxymethyl]- thiophene |
| 5 | 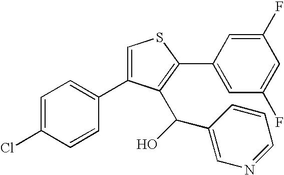 | 4-(4-Chlorophenyl)-2-(3,5- difluorophenyl)-3-[(3- pyridyl)hydroxymethyl]- thiophene |
| 6 | 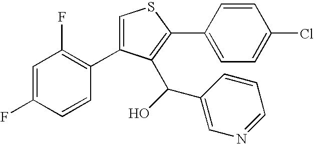 | 2-(4-Chlorophenyl)-4-(2,4- difluorophenyl)-3-[(3- pyridyl)hydroxymethyl]- thiophene |
| 7 | 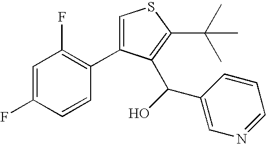 | 4-(2,4-Difluorophenyl)-2-(1,1- dimethylethyl)-3-[(3- pyridyl)hydroxymethyl]- thiophene |
| 8 | 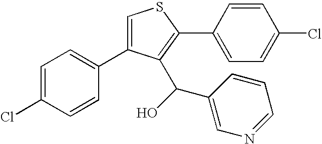 | 2,4-Bis-(4-chlorophenyl)-3-[(3- pyridyl)hydroxymethyl]- thiophene |
| 9 | 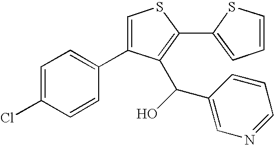 | 4-(4-Chlorophenyl)-3-[(3- pyridyl)hydroxymethyl]-2-(2- thienyl)thiophene |
| 10 | 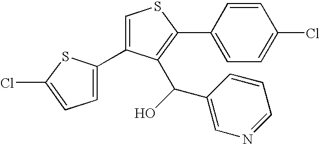 | 2-(4-Chlorophenyl)-4-(5- chloro-2-thienyl)-3-[(3- pyridyl)hydroxymethyl]- thiophene |
| 11 | 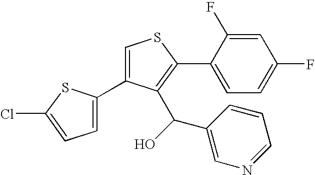 | 4-(5-Choro-2-thienyl)-2-(2,4- difluorophenyl)-3-[(3- pyridyl)hydroxymethyl]- thiophene |
| 12 | 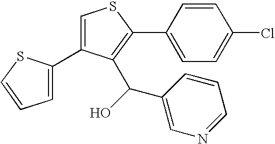 | 2-(4-Chlorophenyl)-3-[(3- pyridyl)hydroxymethyl]-4-(2- thienyl)thiophene |
| 13 | 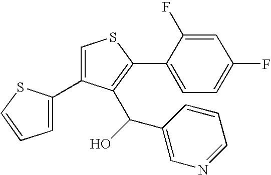 | 2-(2,4-Difluorophenyl)-3-[(3- pyridyl)hydroxymethyl]-4-(2- thienyl)thiophene |
| 14 | 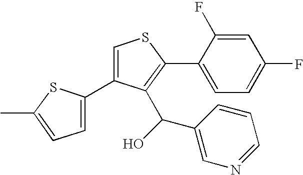 | 2-(2,4-Difluorophenyl)-4-(5- methyl-2-thienyl)-3-[(3- pyridyl)hydroxymethyl]- thiophene |
| 15 | 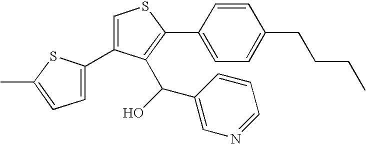 | 2-(4-Butylphenyl)-4-(5-methyl- 2-thienyl)-3-[(3- pyridyl)hydroxymethyl]- thiophene |
| 16 | 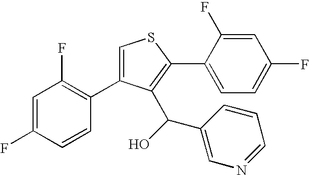 | 2,4-Bis-(2,4-Difluorophenyl)-3- [(3-pyridyl)hydroxymethyl]- thiophene |
| 17 | 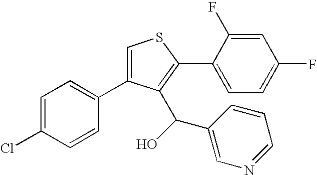 | 4-(4-Chlorophenyl)-2-(2,4- difluorophenyl)-3-[(3- pyridyl)hydroxymethyl]- thiophene |
| 18 | 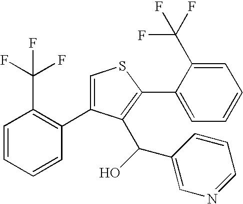 | 2,4-Bis-(2- trifluoromethylphenyl)-3-[(3- pyridyl)hydroxymethyl]- thiophene |
| 19 | 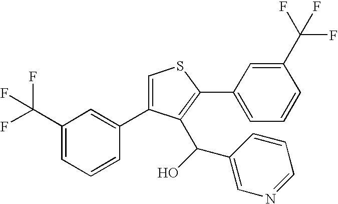 | 2,4-Bis-(3- trifluoromethylphenyl)-3-[(3- pyridyl)hydroxymethyl]- thiophene |
| 20 | 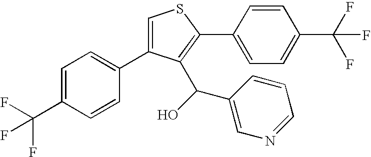 | 2,4-Bis-(4- trifluoromethylphenyl)-3-[(3- pyridyl)hydroxymethyl]- thiophene |
| 21 | 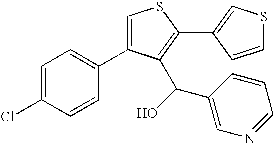 | 4-(4-Chlorophenyl)-3-[(3- pyridyl)hydroxymethyl]-2-(3- thienyl)thiophene |
| 22 | 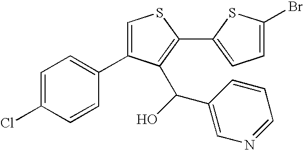 | 2-(5-Bromo-2-thienyl)-4-(4- chlorophenyl)-3-[(3- pyridyl)hydroxymethyl]- thiophene |
| 23 | 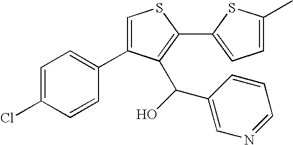 | 4-(4-Chlorophenyl)-2-(5- methyl-2-thienyl)-3-[(3- pyridyl)hydroxymethyl]- thiophene |
| 24 | 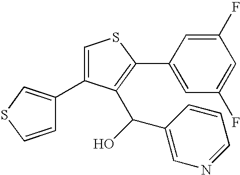 | 2-(3,5-Difluorophenyl)-3-[(3- pyridyl)hydroxymethyl]-4-(3- thienyl)thiophene |
| 25 | 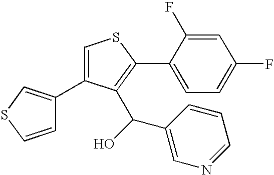 | 2-(2,4-Difluorophenyl)-3-[(3- pyridyl)hydroxymethyI]-4-(3- thienyl)thiophene |
| 26 | 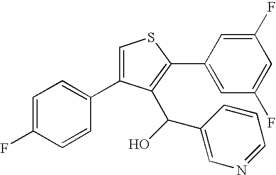 | 2-(3,5-Difluorophenyl)-4-(4- fluorophenyl)-3-[(3- pyridyl)hydroxymethyl]- thiophene |
| 27 | 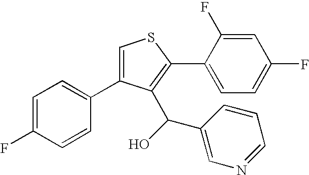 | 2-(2,4-Difluorophenyl)-4-(4- fluorophenyl)-3-[(3- pyridyl)hydroxymethyl]- thiophene |
| 28 | 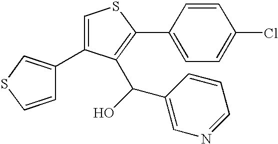 | 2-(4-Chlorophenyl)-3-[(3- pyridyl)hydroxymethyl]-4-(3- thienyl)thiophene |
| 29 | 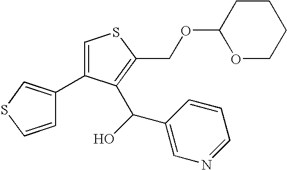 | 3-[(3-Pyridyl)hydroxymethyl]- 2-(2-tetrahydropyranyloxy- methyl)-4-(3-thienyl)thiophene |
| 30 | 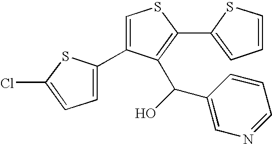 | 4-(5-Chloro-2-thienyl)-3-[(3- pyridyl)hydroxymethyl]-2-(2- thienyl)thiophene |
| 31 | 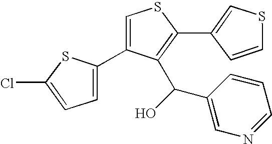 | 4-(5-Chloro-2-thienyl)-3-[(3- pyridyl)hydroxymethyl]-2-(3- thienyl)thiophene |
| 32 | 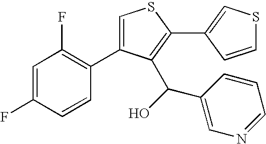 | 4-(2,4-Difluorophenyl)-3-[(3- pyridyl)hydroxymethyl]-2-(3- thienyl)thiophene |
| 33 | 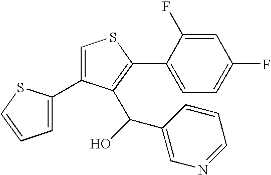 | 2-(2,4-Difluorophenyl)-3-[(3- pyridyl)hydroxymethyl]-4-(2- thienyl)thiophene |
| 34 | 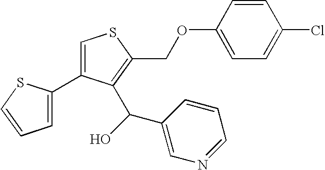 | 2-(4-Chlorophenoxymethyl)-3- [(3-pyridyl)hydroxymethyl]-4- (2-thienyl)thiophene |
| 35 | 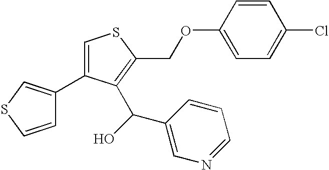 | 2-(4-Chlorophenoxymethyl)-3- [(3-pyridyl)hydroxymethyl]-4- (3-thienyl)thiophene |
| 36 | 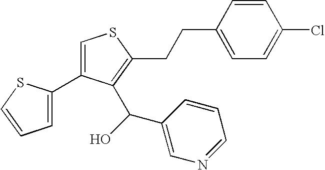 | 2-(4-Chlorophenylethyl)-3-[(3- pyridyl)hydroxymethyl]-4-(2- thienyl)thiophene |
| 37 | 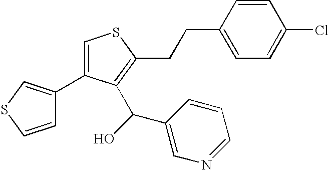 | 2-(4-Chlorophenylethyl)-3-[(3- pyridyl)hydroxymethyl]-4-(3- thienyl)thiophene |
| 38 | 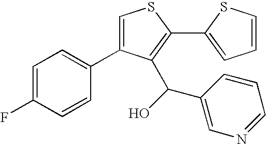 | 4-(4-Fluorophenyl)-3-[(3- pyridyl)hydroxymethyl]-2-(2- thienyl)thiophene |
| 39 | 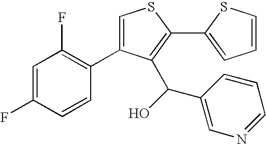 | 4-(2,4-Difluorophenyl)-3-[(3- pyridyl)hydroxymethyl]-2-(2- thienyl)thiophene |
| 40 | 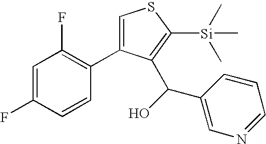 | 4-(2,4-Difluorophenyl)-3-[(3- pyridyl)hydroxymethyl]-2- (trimethylsilyl)thiophene |
| 41 | 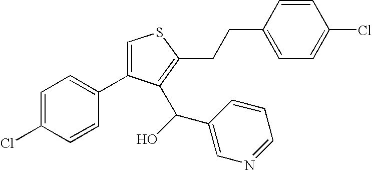 | 4-(4-Chlorophenyl)-2-(4- chlorophenylethyl)-3-[(3- pyridyl)hydroxymethyl]- thiophene |
| 42 | 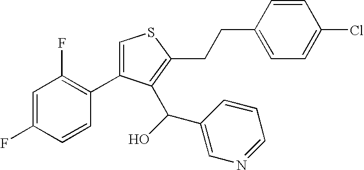 | 2-(4-Chlorophenylethyl)-4- (2,4-difluorophenyl)-3-[(3- pyridyl)hydroxymethyl]- thiophene |
| 43 | 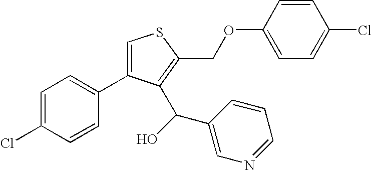 | 2-(4-Chlorophenoxymethyl)-4- (4-chlorophenyl)-3-[(3- pyridyl)hydroxymethyl]- thiophene |
| 44 | 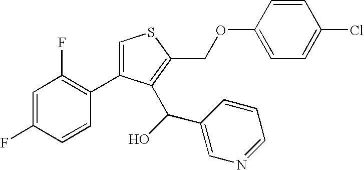 | 2-(4-Chlorophenoxymethyl)-4- (2,4-difluorophenyl)-3-[(3- pyridyl)hydroxymethyl]- thiophene |
| 45 | 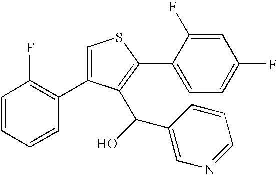 | 2-(2,4-Difluorophenyl)-4-(2- fluorophenyl)-3-[(3- pyridyl)hydroxymethyl]- thiophene |
| 46 | 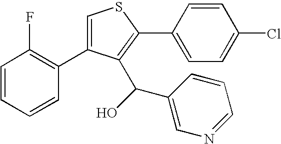 | 2-(4-Chlorophenyl)-4-(2- fluorophenyl)-3-[(3- pyridyl)hydroxymethyl]- thiophene |
| 47 | 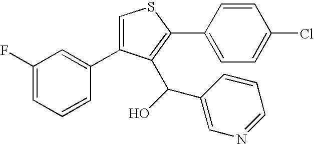 | 2-(4-Chlorophenyl)-4-(3- fluorophenyl)-3-[(3- pyridyl)hydroxymethyl]- thiophene |
| 48 | 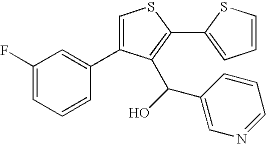 | 4-(3-Fluorophenyl)-3-[(3- pyridyl)hydroxymethyl]-2-(2- thienyl)thiophene |
| 49 | 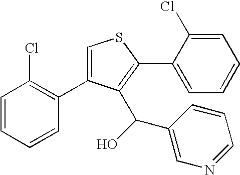 | 2,4-Bis-(2-Chlorophenyl)-3-[(3- pyridyl)hydroxymethyl]- thiophene |
| 50 | 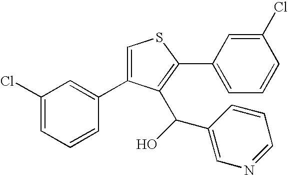 | 2,4-Bis-(3-Chlorophenyl)-3-[(3- pyridyl)hydroxymethyl]- thiophene |
| 51 | 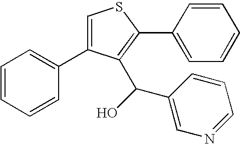 | 2,4-Bis-(Phenyl)-3-[(3- pyridyl)hydroxymethyl]- thiophene |
| 52 | 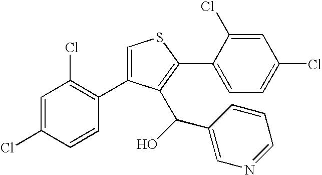 | 2,4-Bis-(2,4-Dichlorophenyl)- 3-[(3-pyridyl)hydroxymethyl]- thiophene |
| 53 | 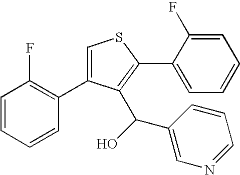 | 2,4-Bis-(2-FluorophenyD-3-[(3- pyridyl)hydroxymethyl]- thiophene |
| 54 | 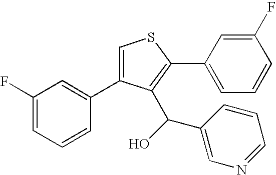 | 2,4-Bis-(3-Fluorophenyl)-3-[(3- pyridyl)hydroxymethyl]- thiophene |
| 55 | 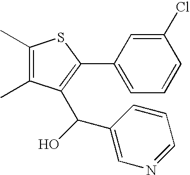 | 2-(3-Chlorophenyl)-4,5- dimethyl-3-[(3- pyridyl)hydroxymethyl]- thiophene |
| 56 | 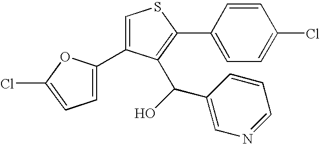 | 4-(5-Chloro-2-furanyl)-2-(4- chlorophenyl)-3-[(3- pyridyl)hydroxymethyl]- thiophene |
| 57 | 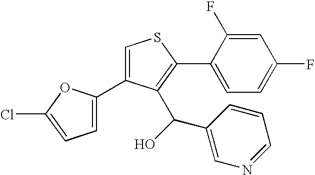 | 4-(5-Chloro-2-furanyl)-2-(2,4- difluorophenyl)-3-[(3- pyridyl)hydroxymethyl]- thiophene |
| 58 | 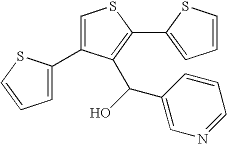 | 2,4-Bis-(2-thienyl)-3-[(3- pyridyl)hydroxymethyl]- thiophene |
| 59 | 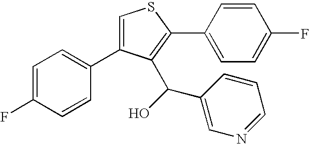 | 2,4-Bis-(4-fluorophenyl)-3-[(3- pyridyl)hydroxymethyl]- thiophene |
| 60 | 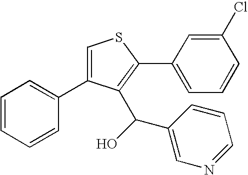 | 2-(3-Chlorophenyl)-4-phenyl- 3-[(3-pyridyl)hydroxymethyl]- thiophene |
| 61 | 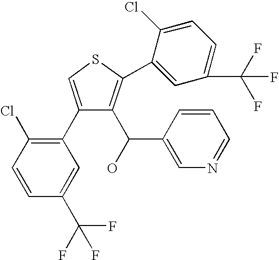 | 2,4-Bis-(3-chloro-5- trifluoromethylphenyl)-3-[(3- pyridyl)hydroxymethyl]- thiophene |
| 62 | 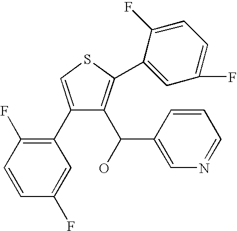 | 2,4-Bis-(2,5-difluorophenyl)-3- [(3-pyridyl)hydroxymethyl]- thiophene |
| 63 | 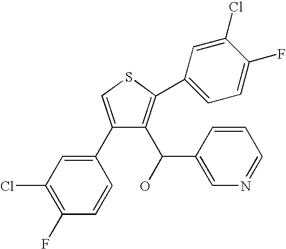 | 2,4-Bis-(4-chloro-3- fluorophenyl)-3-[(3- pyridyl)hydroxymethyl]- thiophene |
| 64 | 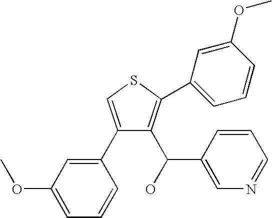 | 2,4-Bis-(3-Methoxyphenyl)-3- [(3-pyridyl)hydroxymethyl]- thiophene |
| 65 | 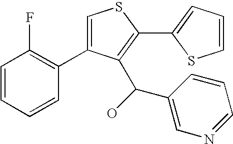 | 4-(2-Fluorophenyl)-3-[(3- pyridyl)hydroxymethyl]-2-(2- thienyl)thiophene |
| 66 | 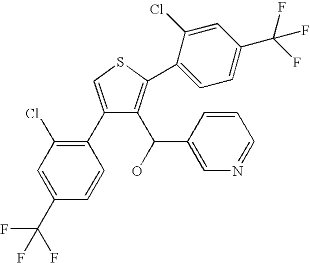 | 2,4-Bis-(2-chloro-4- trifluoromethylphenyl)-3-[(3- pyridyl)hydroxymethyl]- thiophene |
| 67 | 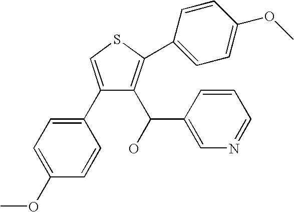 | 2,4-Bis-(4-Methoxyphenyl)-3- [(3-pyridyl)hydroxymethyl]- thiophene |
| 68 | 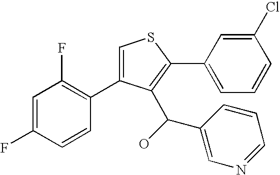 | 2-(3-Chlorophenyl)-4-(2,4- difluorophenyl)-3-[(3- pyridyl)hydroxymethyl]- thiophene |
| 69 | 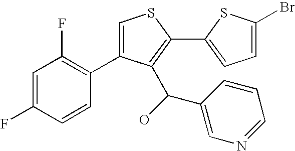 | 2-(5-Bromo-2-thienyl)-4-(2,4- difluorophenyl)-3-[(3- pyridyl)hydroxymethyl]- thiophene |
| 70 | 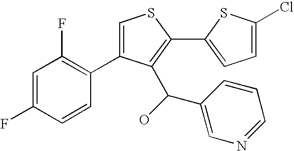 | 2-(5-Chloro-2-thienyl)-4-(2,4- difluorophenyl)-3-[(3- pyridyl)hydroxymethyl]- thiophene |
| 71 | 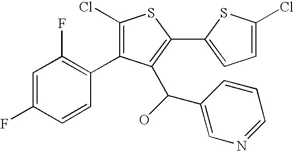 | 5-Chloro-2-(5-chloro-2- thienyl)-4-(2,4-difluorophenyl)- 3-[(3-pyridyl)hydroxymethyl]- thiophene |
| 72 | 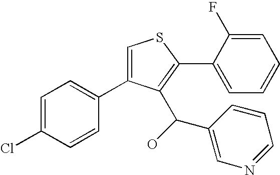 | 4-(4-Chlorophenyl)-2-(2- fluorophenyl)-3-[(3- pyridyl)hydroxymethyl]- thiophene |
| 73 | 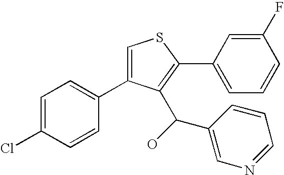 | 4-(4-Chlorophenyl)-2-(3- fluorophenyl)-3-[(3- pyridyl)hydroxymethyl]- thiophene |
| 74 | 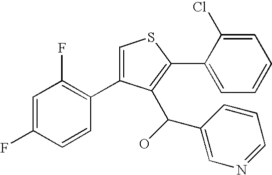 | 2-(2-Chlorophenyl)-4-(2,4- difluorophenyl)-3-[(3- pyridyl)hydroxymethyl]- thiophene |
| 75 | 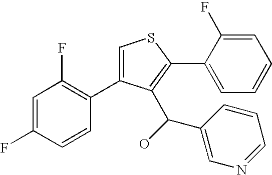 | 4-(2,4-Difluorophenyl)-2-(2- fluorophenyl)-3-[(3- pyridyl)hydroxymethyl]- thiophene |
| 76 | 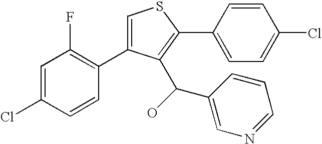 | 2-(4-Chlorophenyl)-4-(4- chloro-2-fluorophenyl)-3-[(3- pyridyl)hydroxymethyl]- thiophene |
| 77 | 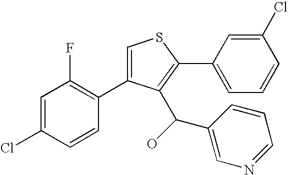 | 2-(3-Chlorophenyl)-4-(4- chloro-2-fluorophenyl)-3-[(3- pyridyl)hydroxymethyl]- thiophene |
| 78 | 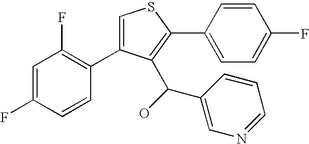 | 4-(2,4-Difluorophenyl)-2-(4- fluorophenyl)-3-[(3- pyridyl)hydroxymethyl]- thiophene |
| 79 | 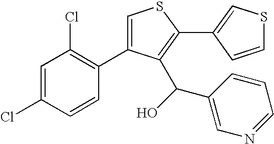 | 4-(2,4-Dichlorophenyl)-3-[(3- pyridyl)hydroxymethyl]-2-(3- thienyl)thiophene |
| 80 | 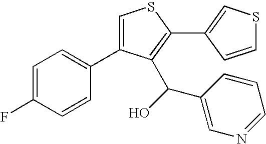 | 4-(4-Fluorophenyl)-3-[(3- pyridyl)hydroxymethyl]-2-(3- thienyl)thiophene |
| 81 | 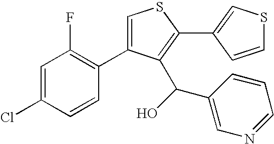 | 4-(4-Chloro-2-fluorophenyl)-3- [(3-pyridyl)hydroxymethyl]-2- (3-thienyl)thiophene |
| 82 | 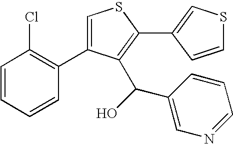 | 4-(2-Chlorophenyl)-3-[(3- pyridyl)hydroxymethyl]-2-(3- thienyl)thiophene |
| 83 | 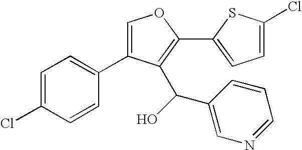 | 4-(4-Chlorophenyl)-2-(5- chloro-2-thienyl)-3-[(3- pyridyl)hydroxymethyl]furan |
| 84 | 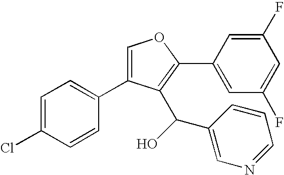 | 4-(4-Chlorophenyl)-2-(3,5- difluorophenyl)-3-[(3- pyridyl)hydroxymethyl]furan |
| 85 | 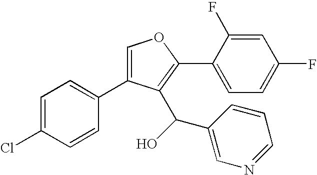 | 4-(4-Chlorophenyl)-2-(2,4- difluorophenyl)-3-[(3- pyridyl)hydroxymethyl]furan |
| 86 | 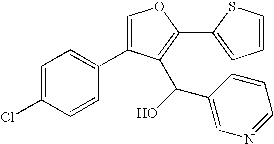 | 4-(4-Chlorophenyl)-3-[(3- pyridyl)hydroxymethyl]-2-(2- thienyl)furan |
| 87 | 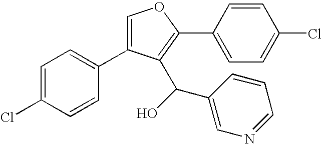 | 2,4-Bis-(4-chlorophenyl)-3-[(3- pyridyl)hydroxymethyl]furan |
| 88 | 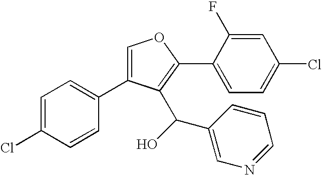 | 4-(4-Chlorophenyl)-2-(4- chloro-2-fluorophenyl)-3-[(3- pyridyl)hydroxymethyl]furan |
| 89 | 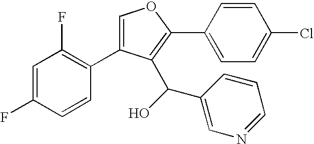 | 2-(4-Chlorophenyl)-4-(2,4- difluorophenyl)-3-[(3- pyridyl)hydroxymethyl]furan |
| 90 | 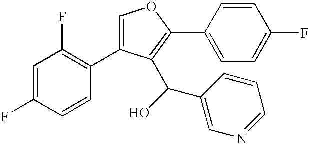 | 4-(2,4-Difluorophenyl)-2-(4- fluorophenyl)-3-[(3- pyridyl)hydroxymethyl]furan |
| 91 | 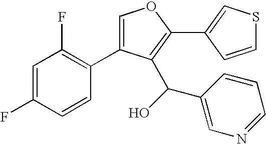 | 4-(2,4-Difluorophenyl)-3-[(3- pyridyl)hydroxymethyl]-2-(3- thienyl)furan |
| 92 | 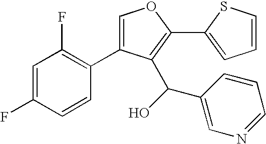 | 4-(2,4-Difluorophenyl)-3-[(3- pyridyl)hydroxymethyl]-2-(2- thienyl)furan |
| 93 | 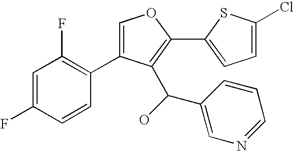 | 2-(5-Chloro-2-thienyl)-4-(2,4- difluorophenyl)-3-[(3- pyridyl)hydroxymethyl]furan |
| 94 | 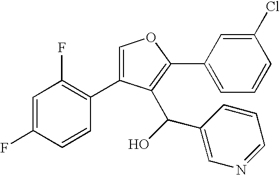 | 2-(3-Chlorophenyl)-4-(2,4- difluorophenyl)-3-[(3- pyridyl)hydroxymethyl]furan |
| 95 | 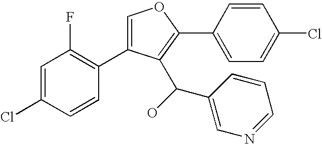 | 2-(4-Chlorophenyl)-4-(4- chloro-2-fluorophenyl)-3-[(3- pyridyl)hydroxymethyl]furan |
| 96 | 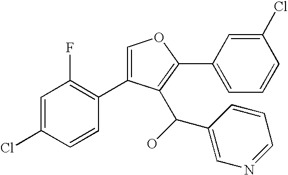 | 2-(3-Chlorophenyl)-4-(4- chloro-2-fluorophenyl)-3-[(3- pyridyl)hydroxymethyl]furan |
| 97 | 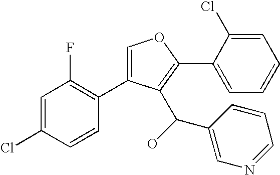 | 2-(2-Chlorophenyl)-4-(4- chloro-2-fluorophenyl)-3-[(3- pyridyl)hydroxymethyl]furan |
| 98 | 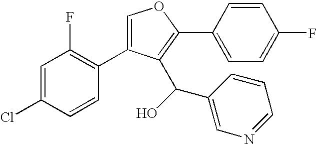 | 4-(4-Chloro-2-fluorophenyl)-2- (4-fluorophenyl)-3-[(3- pyridyl)hydroxymethyl]furan |
| 99 | 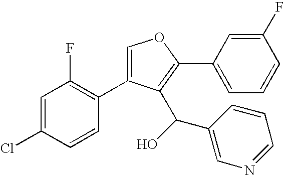 | 4-(4-Chloro-2-fluorophenyl)-2- (3-fluorophenyl)-3-[(3- pyridyl)hydroxymethyl]furan |
| 100 | 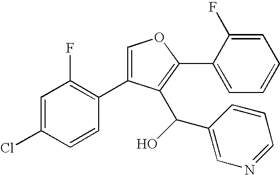 | 4-(4-Chloro-2-fluorophenyl)-2- (2-fluorophenyl)-3-[(3- pyridyl)hydroxymethyl]furan |
| 101 | 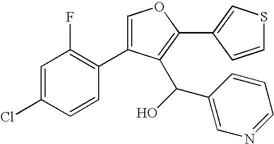 | 4-(4-Chloro-2-fluorophenyl)-3- [(3-pyridyl)hydroxymethyl]-2- (3-thienyl)furan |
| 102 | 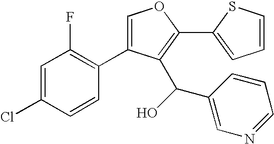 | 4-(4-Chloro-2-fluorophenyl)-3- [(3-pyridyl)hydroxymethyl]-2- (2-thienyl)furan |
| 103 | 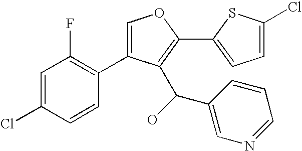 | 4-(4-Chloro-2-fluorophenyl)-2- (5-chloro-2-thienyl)-3-[(3- pyridyl)hydroxymethyl]furan |
| 104 | 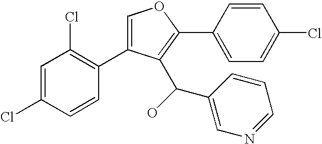 | 2-(4-Chlorophenyl)-4-(2,4- dichlorophenyl)-3-[(3- pyridyl)hydroxymethyl]furan |
| 105 | 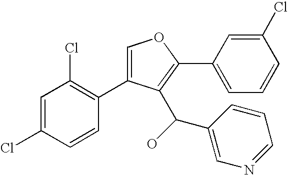 | 2-(3-Chlorophenyl)-4-(2,4- dichlorophenyl)-3-[(3- pyridyl)hydroxymethyl]furan |
| 106 | 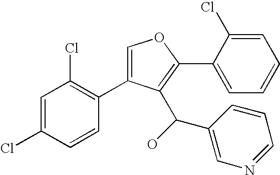 | 2-(2-Chlorophenyl)-4-(2,4- dichlorophenyl)-3-[(3- pyridyl)hydroxymethyl]furan |
| 107 | 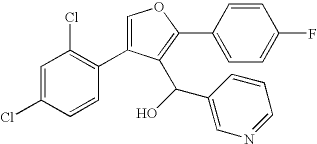 | 4-(2,4-DichIorophenyl)-2-(4- fluorophenyl)-3-[(3- pyridyl)hydroxymethyl]furan |
| 108 | 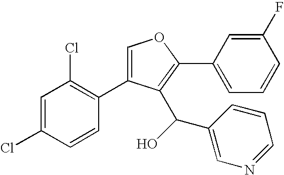 | 4-(2,4-Dichlorophenyl)-2-(3- fluorophenyl)-3-[(3- pyridyl)hydroxymethyl]furan |
| 109 | 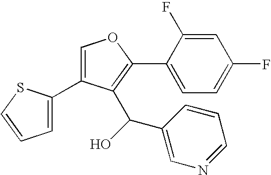 | 2-(2,4-Difluorophenyl)-3-[(3- pyridyl)hydroxymethyl]-4-(2- thienyl)furan |
| 110 | 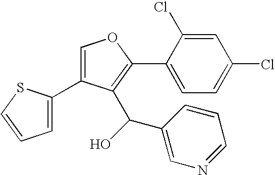 | 2-(2,4-Dichlorophenyl)-3-[(3- pyridyl)hydroxymethyl]-4-(2- thienyl)furan |
| 111 | 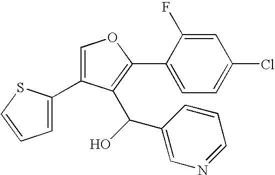 | 2-(4-Chloro-2-fluorophenyl)-3- [(3-pyridyl)hydroxymethyl]-4- (2-thienyl)furan |
| 112 | 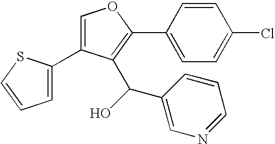 | 2-(4-Chlorophenyl)-3-[(3- pyridyl)hydroxymethyl]-4-(2- thienyl)furan |
| 113 | 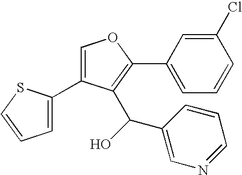 | 2-(3-Chlorophenyl)-3-[(3- pyridyl)hydroxymethyl]-4-(2- thienyl)furan |
| 114 | 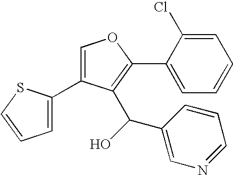 | 2-(2-Chlorophenyl)-3-[(3- pyridyl)hydroxymethyl]-4-(2- thienyl)furan |
| 115 | 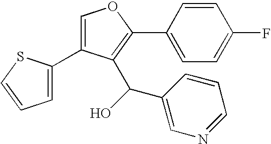 | 2-(4-Fluorophenyl)-3-[(3- pyridyl)hydroxymethyl]-4-(2- thienyl)furan |
| 116 | 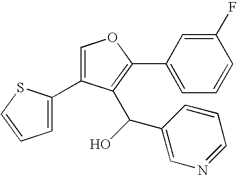 | 2-(3-Fluorophenyl)-3-[(3- pyridyl)hydroxymethyl]-4-(2- thienyl)furan |
| 117 | 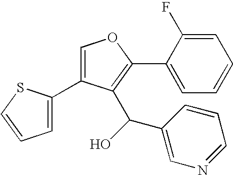 | 2-(2-Fluorophenyl)-3-[(3- pyridyl)hydroxymethyl]-4-(2- thienyl)furan |
| 118 | 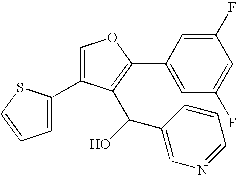 | 2-(3,5-Difluorophenyl)-3-[(3- pyridyl)hydroxymethyl]-4-(2- thienyl)furan |
| 119 | 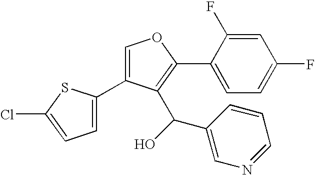 | 4-(5-Chloro-2-thienyl)-2-(2,4- difluorophenyl)-3-[(3- pyridyl)hydroxymethyl]furan |
| 120 | 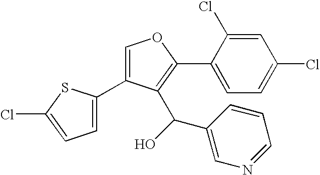 | 4-(5-Chloro-2-thienyl)-2-(2,4- dichlorophenyl)-3-[(3- pyridyl)hydroxymethyl]furan |
| 121 | 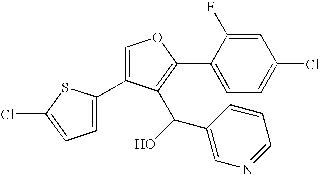 | 2-(4-Chloro-2-fluorophenyl)-4- (5-chloro-2-thienyl)-3-[(3- pyridyl)hydroxymethyl]furan |
| 122 | 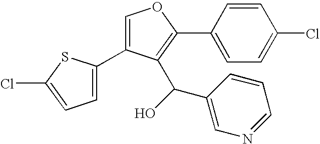 | 2-(4-Chlorophenyl)-4-(5- chloro-2-thienyl)-3-[(3- pyridyl)hydroxymethyl]furan |
| 123 | 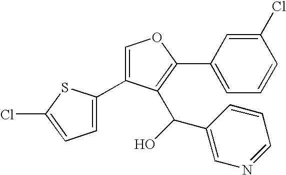 | 2-(3-Chlorophenyl)-4-(5- chloro-2-thienyl)-3-[(3- pyridyl)hydroxymethyl]furan |
| 124 | 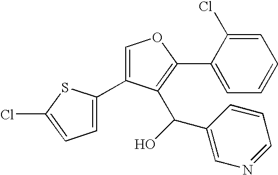 | 2-(2-Chlorophenyl)-4-(5- chloro-2-thienyl)-3-[(3- pyridyl)hydroxymethyl]furan |
| 125 | 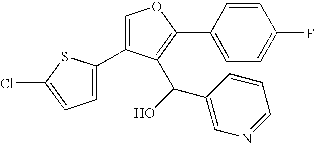 | 4-(5-Chloro-2-thienyl)-2-(4- fluorophenyl)-3-[(3- pyridyl)hydroxymethyl]furan |
| 126 | 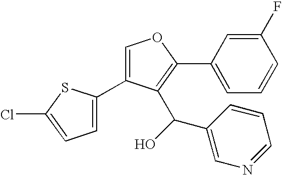 | 4-(5-Chloro-2-thienyl)-2-(3- fluorophenyl)-3-[(3- pyridyl)hydroxymethyl]furan |
| 127 | 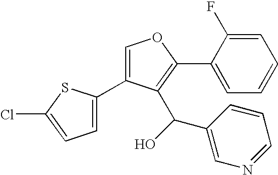 | 4-(5-Chloro-2-thienyl)-2-(2- fluorophenyl)-3-[(3- pyridyl)hydroxymethyl]furan |
| 128 | 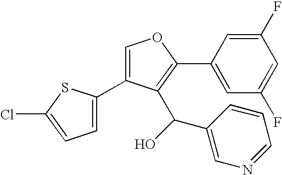 | 4-(5-Chloro-2-thienyl)-2-(3,5- difluorophenyl)-3-[(3- pyridyl)hydroxymethyl]furan |
| 129 | 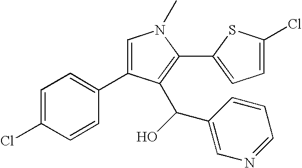 | 4-(4-Chlorophenyl)-2-(5- chloro-2-thienyl)-1-(N-methyl)- 3-[(3- pyridyl)hydroxymethyl]pyrrole |
| 130 | 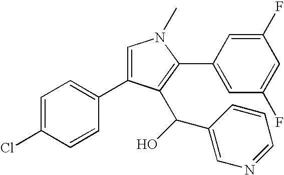 | 4-(4-Chlorophenyl)-2-(3,5- difluorophenyl)-1-(N-methyl)- 3-[(3- pyridyl)hydroxymethyl]pyrrole |
| 131 | 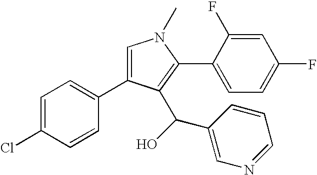 | 4-(4-Chlorophenyl)-2-(2,4- difluorophenyl)-1-(N-methyl)- 3-[(3- pyridyl)hydroxymethyl]pyrrole |
| 132 | 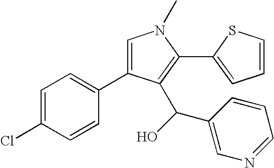 | 4-(4-Chlorophenyl)-1-(N- methyl)-3-[(3- pyridyl)hydroxymethyl]-2-(2- thienyl)pyrrole |
| 133 | 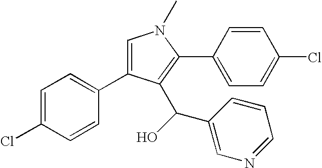 | 2,4-Bis-(4-chlorophenyl)-1-(N- methyl)-3-[(3- pyridyl)hydroxymethyl]pyrrole |
| 134 | 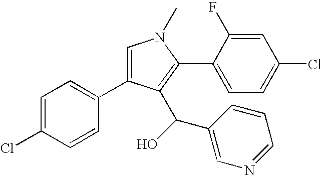 | 4-(4-Chlorophenyl)-2-(4- chloro-2-fluorophenyl)-3-[(3- pyridyl)hydroxymethyl]pyrrole |
| 135 | 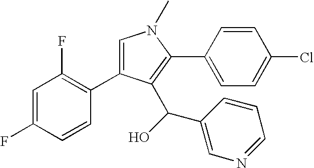 | 2-(4-Chlorophenyl)-4-(2,4- difluorophenyl)-1-(N-methyl)- 3-[(3- pyridyl)hydroxymethyl]pyrrole |
| 136 | 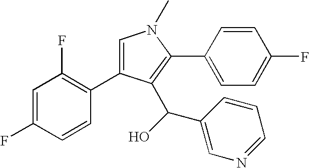 | 4-(2,4-Difluorophenyl)-2-(4- fluorophenyl)-1-(N-methyl)-3- [(3- pyridyl)hydroxymethyl]pyrrole |
| 137 | 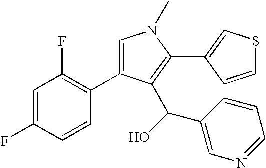 | 4-(2,4-Difluorophenyl)-1-(N- methyl)-3-[(3- pyridyl)hydroxymethyl]-2-(3- thienyl)pyrrole |
| 138 | 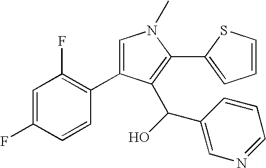 | 4-(2,4-Difluorophenyl)-1-(N- methyl)-3-[(3- pyridyl)hydroxymethyl]-2-(2- thienyl)pyrrole |
| 139 | 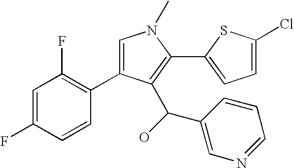 | 2-(5-Chloro-2-thienyl)-4-(2,4- difluorophenyl)-1-(N-methyl)- 3-[(3- pyridyl)hydroxymethyl]pyrrole |
| 140 | 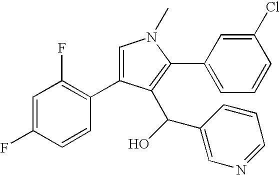 | 2-(3-Chlorophenyl)-4-(2,4- difluorophenyl)-1-(N-methyl)- 3-[(3- pyridyl)hydroxymethyl]pyrrole |
| 141 | 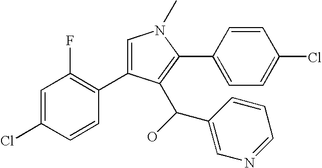 | 2-(4-Chlorophenyl)-4-(4- chloro-2-fluorophenyl)-1-(N- methyl)-3-[(3- pyridyl)hydroxymethyl]pyrrole |
| 142 | 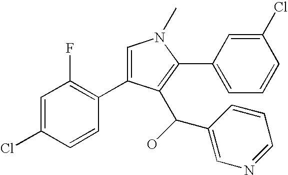 | 2-(3-Chlorophenyl)-4-(4- chloro-2-fluorophenyl)-1-(N- methyl)-3-[(3- pyridyl)hydroxymethyl]pyrrole |
| 143 | 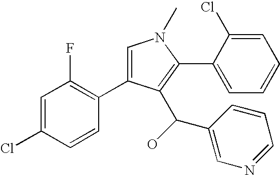 | 2-(2-Chlorophenyl)-4-(4- chloro-2-fluorophenyl)-1-(N- methyl)-3-[(3- pyridyl)hydroxymethyl]pyrrole |
| 144 | 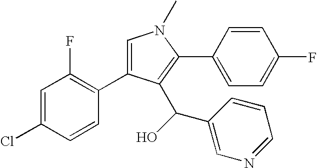 | 4-(4-Chloro-2-fluorophenyl)-2- (4-fluorophenyl)-1-(N-methyl)- 3-[(3- pyridyl)hydroxymethyl]pyrrole |
| 145 | 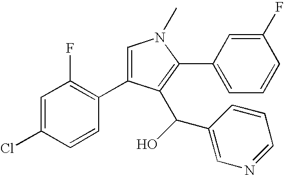 | 4-(4-Chloro-2-fluorophenyl)-2- (3-fluorophenyl)-1-(N-methyl)- 3-[(3- pyridyl)hydroxymethyl]pyrrole |
| 146 | 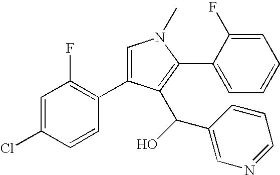 | 4-(4-Chloro-2-fluorophenyl)-2- (2-fluorophenyl)-1-(N-methyl)- 3-[(3- pyridyl)hydroxymethyl]pyrrole |
| 147 | 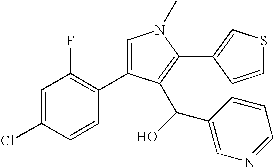 | 4-(4-Chloro-2-fluorophenyl)-1- (N-methyl)-3-[(3- pyridyl)hydroxymethyl]-2-(3- thienyl)pyrrole |
| 148 | 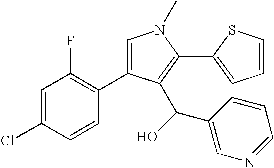 | 4-(4-Chloro-2-fluorophenyl)-1- (N-methyl)-3-[(3- pyridyl)hydroxymethyl]-2-(2- thienyl)pyrrole |
| 149 | 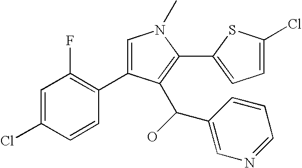 | 4-(4-Chloro-2-fluorophenyl)-2- (5-chloro-2-thienyl)-1-(N- methyl)-3-[(3- pyridyl)hydroxymethyl]pyrrole |
| 150 | 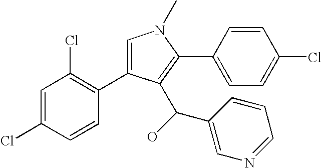 | 2-(4-Chlorophenyl)-4-(2,4- dichlorophenyl)-1-(N-methyl)- 3-[(3- pyridyl)hydroxymethyl]pyrrole |
| 151 | 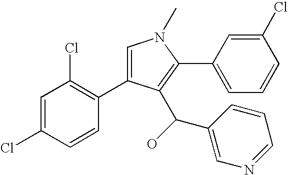 | 2-(3-Chlorophenyl)-4-(2,4- dichlorophenyl)-1-(N-methyl)- 3-[(3- pyridyl)hydroxymethyl]pyrrole |
| 152 | 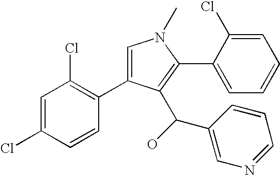 | 2-(2-Chlorophenyl)-4-(2,4- dichlorophenyl)-1-(N-methyl)- 3-[(3- pyridyl)hydroxymethyl]pyrrole |
| 153 | 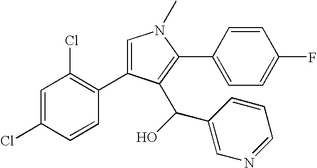 | 4-(2,4-Dichlorophenyl)-2-(4- fluorophenyl)-1-(N-methyl)-3- [(3- pyridyl)hydroxymethyl]pyrrole |
| 154 | 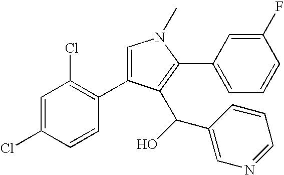 | 4-(2,4-Dichlorophenyl)-2-(3- fluorophenyl)-1-(N-methyl)-3- [(3- pyridyl)hydroxymethyl]pyrrole |
| 155 | 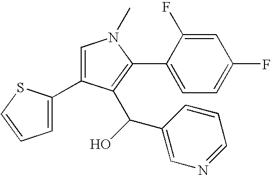 | 2-(2,4-Difluorophenyl)-1-(N- methyl)-3-[(3- pyridyl)hydroxymethyl]-4-(2- thienyl)pyrrole |
| 156 | 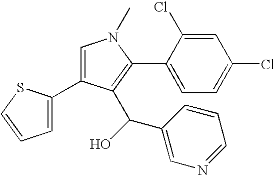 | 2-(2,4-Dichlorophenyl)-1-(N- methyl)-3-[(3- pyridyl)hydroxymethyl]-4-(2- thienyl)pyrrole |
| 157 | 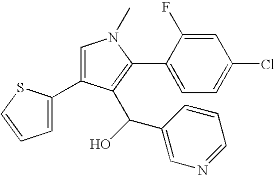 | 2-(4-Chloro-2-fluorophenyl)-1- (N-methyl)-3-[(3- pyridyl)hydroxymethyl]-4-(2- thienyl)pyrrole |
| 158 | 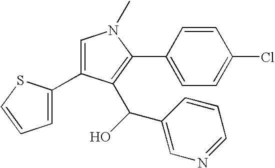 | 2-(4-Chlorophenyl)-1-(N- methyl)-3-[(3- pyridyl)hydroxymethyl]-4-(2- thienyl)pyrrole |
| 159 | 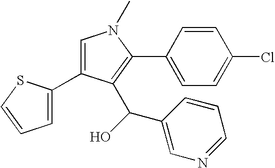 | 2-(3-Chlorophenyl)-1-(N- methyl)-3-[(3- pyridyl)hydroxymethyl]-4-(2- thienyl)pyrrole |
| 160 | 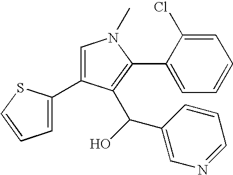 | 2-(2-Chlorophenyl)-1-(N- methyl)-3-[(3- pyridyl)hydroxymethyl]-4-(2- thienyl)pyrrole |
| 161 | 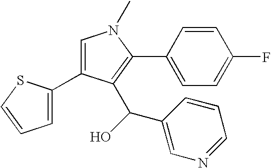 | 2-(4-Fluorophenyl)-1-(N- methyl)-3-[(3- pyridyl)hydroxymethyl]-4-(2- thienyl)pyrrole |
| 162 | 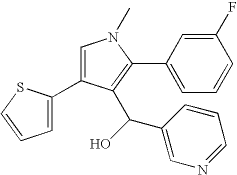 | 2-(3-Fluorophenyl)-1-(N- methyl)-3-[(3- pyridyl)hydroxymethyl]-4-(2- thienyl)pyrrole |
| 163 | 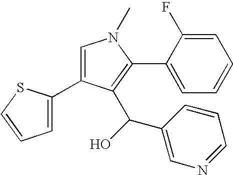 | 2-(2-Fluorophenyl)-1-(N- methyl)-3-[(3- pyridyl)hydroxymethyl]-4-(2- thienyl)pyrrole |
| 164 | 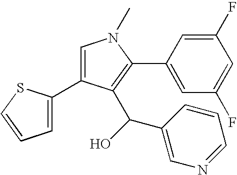 | 2-(3,5-Difiuorophenyl)-1-(N- methyl)-3-[(3- pyridyl)hydroxymethyl]-4-(2- thienyl)pyrrole |
| 165 | 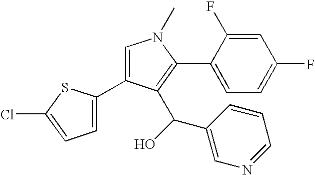 | 4-(5-Chloro-2-thienyl)-2-(2,4- difluorophenyl)-1-(N-methyl)- 3-[(3- pyridyl)hydroxymethyl]pyrrole |
| 166 | 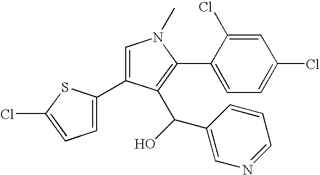 | 4-(5-Chloro-2-thienyl)-2-(2,4- dichlorophenyl)-1-(N-methyl)- 3-[(3- pyridyl)hydroxymethyl]pyrrole |
| 167 | 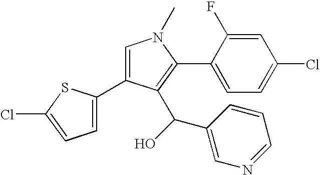 | 2-(4-Chloro-2-fluorophenyl)-4- (5-chloro-2-thienyl)-1-(N- methyl)-3-[(3- pyridyl)hydroxymethyl]pyrrole |
| 168 | 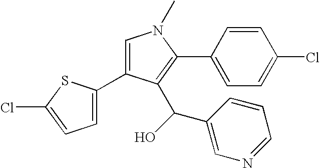 | 2-(4-Chlorophenyl)-4-(5- chloro-2-thienyl)-1-(N-methyl)- 3-[(3- pyridyl)hydroxymethyl]pyrrole |
| 169 | 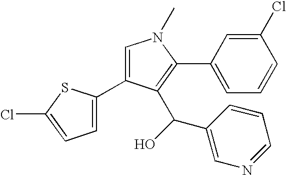 | 2-(3-Chlorophenyl)-4-(5- chloro-2-thienyl)-1-(N-methyl)- 3-[(3- pyridyl)hydroxymethyl]pyrrole |
| 170 | 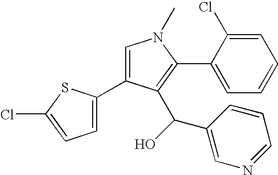 | 2-(2-Chlorophenyl)-4-(5- chloro-2-thienyl)-1-(N-methyl)- 3-[(3- pyridyl)hydroxymethyl]pyrrole |
| 171 | 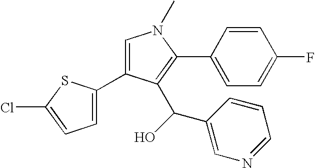 | 4-(5-Chloro-2-thienyl)-2-(4- fluorophenyl)-1-(N-methyl)-3- [(3- pyridyl)hydroxymethyl]pyrrole |
| 172 | 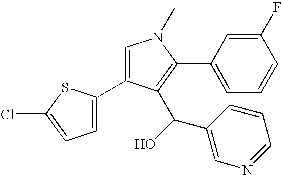 | 4-(5-Chloro-2-thienyl)-2-(3- fluorophenyl)-1-(N-methyl)-3- [(3- pyridyl)hydroxymethyl]pyrrole |
| 173 | 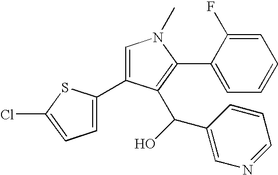 | 4-(5-Chloro-2-thienyl)-2-(2- fluorophenyl)-1-(N-methyl)-3- [(3- pyridyl)hydroxymethyl]pyrrole |
| 174 | 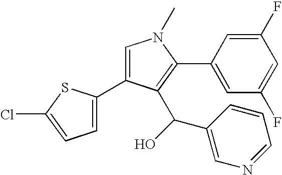 | 4-(5-Chloro-2-thienyl)-2-(3,5- difluorophenyl)-1-(N-methyl)- 3-[(3- pyridyl)hydroxymethyl]pyrrole |

Salts. The compounds described herein and, optionally, all their isomers may be obtained in the form of their salts. Because some of the compounds I have a basic canter they can, for example, form acid addition salts. Said acid addition salts are, for example, formed with mineral acids, typically sulfuric acid, a phosphoric acid or a hydrogen halide, with organic carboxylic acids, typically acetic acid, oxalic acid, malonic acid, fumaric acid or phthalic acid, with hydroxycarboxylic acids, typically ascorbic acid, lactic acid, malic acid, tartaric acid or citric acid, or with benzoic acid, or with organic sulfonic acids, typically methanesulfonic acid or p-toluensulfonic acid. Together with at least one acidic group, the compounds of formula I can also form salts with bases. Suitable salts with bases are, for example, metal salts, tyically alkali metal salts; or alkaline earth metal salts, e.g. sodium salts, potassium salts or magnesium salts, or salts with ammonia or an organic amine, e.g. morpholine, piperidine, pyrrolidine, a mono-, di- or trialkylamine, typically ethylamine, diethylamine, triethylamine or dimethylpropylamine, or a mono-, di- or trihydroxyalkylamine, typically mono-, di- or triethanolamine. Where appropriate, the formation of corresponding internal salts is also possible. Within the scope of this invention, agrochemical or pharmaceutically acceptable salts are preferred.

3. Agrochemical compositions and use. Active compounds of the present invention can be used to prepare agrochemical compositions and used to control fungi in like manner as other antifungal compounds. See, e.g., U.S. Pat. No. 6,617,330; see also U.S. Pat. Nos. 6,616,952; 6,569,875; 6,541,500, and 6,506,794.

Active compounds described herein can be used for protecting plants against diseases that are caused by fungi. For the purposes herein, oomycetes shall be considered fungi. The active compounds can be used in the agricultural sector and related fields as active ingredients for controlling plant pests. The active compounds can be used to inhibit or destroy the pests that occur on plants or parts of plants (fruit, blossoms, leaves, stems, tubers, roots) of different crops of useful plants, optionally while at the same time protecting also those parts of the plants that grow later e.g. from phytopathogenic micro-organisms.

Active compounds may be used as dressing agents for the treatment of plant propagation material, in particular of seeds (fruit, tubers, grains) and plant cuttings (e.g. rice), for the protection against fungal infections as well as against phytopathogenic fungi occurring in the soil.

The active compounds may be used, for example, against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they may also be used against the Ascomycetes classes (e.g. Venturia and Erysiphe, Podosphaera, Monilinia, Uncinula) and of the Oomycetes classes (e.g. Phytophthora, Pythium, Plasmopara). Specific examples of fungi that may be treated include, but are not limited to, Septoria tritici, Stagonospora nodorum, Phytophthora infestans, Botrytis cinerea, and Monilinia fructicola.

Target crops to be protected with active compounds and compositions of the invention typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fiber plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamon, camphor) or plants such as tobacco, nuts, coffee, eggplants, sugar cane, tea, pepper, vines including grape-bearing vines, hops, bananas, turf and natural rubber plants, as well as ornamentals (flowers, shrubs, broad-leafed trees and evergreens, such as conifers). This list does not represent any limitation.

The active compounds can be used in the form of compositions and can be applied to the crop area or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides, plant growth regulators, plant activators or mixtures of several of these preparations, if desired together with further carriers, surfactants or application promoting adjuvants customarily employed in the art of formulation.

The active compounds can be mixed with other fungicides, resulting in some cases in unexpected synergistic activities.

Mixing components which are particularly preferred are azoles such as azaconazole, bitertanol, propiconazole, difenoconazole, diniconazole, cyproconazole, epoxiconazole, fluquinconazole, flusilazole, flutriafol, hexaconazole, imazalil, imibenconazole, ipconazole, tebuconazole, tetraconazole, fenbuconazole, metconazole, myclobutanil, perfurazoate, penconazole, bromuconazole, pyrifenox, prochloraz, triadimefon, triadimenol, triflumizole or triticonazole; pyrimidinyl carbinoles such as ancymidol, fenarimol or nuarimol; 2-amino-pyrimidine such as bupirimate, dimethirimol or ethirimol; morpholines such as dodemorph, fenpropidin, fenpropimorph, spiroxamin or tridemorph; anilinopyrimidines such as cyprodinil, pyrimethanil or mepanipyrim; pyrroles such as fenpiclonil or fludioxonil; phenylamides such as benalaxyl, furalaxyl, metalaxyl, R-metalaxyl, ofurace or oxadixyl; benzimidazoles such as benomyl, carbendazim, debacarb, fuberidazole or thiabendazole; dicarboximides such as chlozolinate, dichlozoline, iprodine, myclozoline, procymidone or vinclozolin; carboxamides such as carboxin, fenfuram, flutolanil, mepronil, oxycarboxin or thifluzamide; guanidines such as guazatine, dodine or iminoctadine; strobilurines such as azoxystrobin, kresoxim-methyl, metominostrobin, pyraclostrobin, picoxystrobin, SSF-129, methyl 2[(2-trifluoromethyl)-pyrid-6-yloxymethyl]-3-methoxy-acrylate or 2-[{alpha.[(alpha-methyl-3-trifluoromethyl-benzyl)imino]-oxy}-o-tolyl]-glyoxylic acid-methylester-O-methyloxime (trifloxystrobin); dithiocarbamates such as ferbaam, mancozeb, maneb, metiram, propineb, thiram, zineb or ziram; N-halomethylthio-dicarboximides such as captafol, captan, dichlofluanid, fluoromide, folpet or tolyfluanid; copper compounds such as Bordeaux mixture, copper hydroxide, copper oxychloride, copper sulfate, cuprous oxide, mancopper or oxine-copper; nitrophenol derivatives such as dinocap or nitrothal-isopropyl; organo phosphorous derivatives such as edifenphos, iprobenphos, isoprothiolane, phosdiphen, pyrazophos or toclofos-methyl; and other compounds of diverse structures such as acibenzolar-S-methyl, harpin, anilazine, blasticidin-S, chinomethionat, chloroneb, chlorothalonil, cymoxanil, dichlone, diclomezine, dicloran, diethofencarb, dimethomorph, dithianon, etridiazole, famoxadone, fenamidone, fentin, ferimzone, fluazinam, flusulfamide, fenhexamid, fosetyl-aluminium, hymexazol, kasugamycin, methasulfocarb, pencycuron, phthalide, polyoxins, probenazole, propamocarb, pyroquilon, quinoxyfen, quintozene, sulfur, triazoxide, tricyclazole, triforine, validamycin, (S)-5-methyl-2-methylthio-5-phenyl-3-phenylamino-3,5-di-hydroimidazol-4-on e (RPA 407213), 3,5-dichloro-N-(3-chloro-1-ethyl-1-methyl-2-oxopropyl)-4-methylbenzamide (RH-7281), N-allyl-4,5-dimethyl-2-trimethylsilylthiophene-3-carboxamide (MON 65500), 4-chloro-4-cyano-N,N-dimethyl-5-p-tolylimidazole-1-sulfon-amide (IKF-916), N-(1-cyano-1,2-dimethylpropyl)-2-(2,4-dichlorophenoxy)-propionamide (AC 382042) or iprovalicarb (SZX 722).

The active compounds can be mixed with one or more systemically acquired resistance inducer (“SAR” inducer), alone or in combination with a fungicide as above. SAR inducers are known and described in, for example, U.S. Pat. No. 6,919,298. In general, a SAR inducer is any compound which has the ability to turn on resistance in a plant to a disease-causing agent, including, but not limited to a virus, a bacterium, a fungus, or combinations of these agents. In addition, an SAR inducer may induce resistance to insect feeding in a plant, as defined by Enyedi et al. (1992; Cell 70: 879-886). Exemplary SAR inducers cover many structural families of compounds, but are united by their ability to induce a resistance to plant diseases and/or pest feeding. One class of SAR inducers is the salicylates. The commercial SAR inducers acibenzolar-S-methyl (available as Actigard® from Syngenta), harpin protein (available as Messenger™ from Eden Biosciences), yeast extract hydrolysate from Saccharomyces cerevisiae (available as Keyplex® 350-DP® from Morse Enterprises Limited, Inc. of Miami, Fla.), and Oryzemate are useful in the present invention. Elicitors, including the Goemar products are another class of SAR inducers that can also be used. In addition, ethylene, its biosynthetic precursors, or ethylene releasing compounds such as Ethrel are considered SAR inducers of utility in this context. See also U.S. Pat. No. 6,919,298.

Suitable carriers and adjuvants can be solid or liquid and are substances useful in formulation technology, e.g. natural or regenerated mineral substances, solvents, dispersants, wetting agents, tackifiers, thickeners, binders or fertilizers.

A preferred method of applying an active compound of the invention, or an agrochemical composition which contains at least one of said compounds, is foliar application. The frequency of application and the rate of application will depend on the risk of infestation by the corresponding pathogen. However, the active compounds can also penetrate the plant through the roots via the soil (systemic action) by drenching the locus of the plant with a liquid formulation, or by applying the compounds in solid form to the soil, e.g. in granular form (soil application). In crops of water such as rice, such granulates can be applied to the flooded rice field. The active compounds may also be applied to seeds (coating) by impregnating the seeds or tubers either with a liquid formulation of the fungicide or coating them with a solid formulation.

The term locus as used herein is intended to embrace the fields on which the treated crop plants are growing, or where the seeds of cultivated plants are sown, or the place where the seed will be placed into the soil. The term seed is intended to embrace plant propagating material such as cuttings, seedlings, seeds, and germinated or soaked seeds.

The active compounds are used in unmodified form or, preferably, together with the adjuvants conventionally employed in the art of formulation. To this end they are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomizing, dusting, scattering, coating or pouring, are chosen in accordance with the intended objectives and the prevailing circumstances.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The formulation, i.e. the compositions containing the compound of formula I and, if desired, a solid or liquid adjuvant, are prepared in known manner, typically by intimately mixing and/or grinding the compound with extenders, e.g. solvents, solid carriers and, optionally, surface active compounds (surfactants).

Suitable carriers and adjuvants may be solid or liquid and correspond to the substances ordinarily employed in formulation technology, such as, e.g. natural or regenerated mineral substances, solvents, dispersants, wetting agents, tackifiers, thickeners binding agents or fertilizers. Such carriers are for example described in WO 97/33890.

Further surfactants customarily employed in the art of formulation are known to the expert or can be found in the relevant literature.

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

The compositions may also contain further adjuvants such as stabilizers, antifoams, viscosity regulators, binders or tackifiers as well as fertilizers, micronutrient donors or other formulations for obtaining special effects.

4. Technical materials. The compounds and combinations of the present invention may also be used in the area of controlling fungal infection (particularly by mold and mildew) of technical materials, including protecting technical material against attack of fungi and reducing or eradicating fungal infection of technical materials after such infection has occurred. Technical materials include but are not limited to organic and inorganic materials wood, paper, leather, natural and synthetic fibers, composites thereof such as particle board, plywood, wall-board and the like, woven and non-woven fabrics, construction surfaces and materials, cooling and heating system surfaces and materials, ventilation and air conditioning system surfaces and materials, and the like. The compounds and combinations according the present invention can be applied to such materials or surfaces in an amount effective to inhibit or prevent disadvantageous effects such as decay, discoloration or mold in like manner as described above. Structures and dwellings constructed using or incorporating technical materials in which such compounds or combinations have been applied are likewise protected against attack by fungi.

5. Pharmaceutical uses. In addition to the foregoing, active compounds of the present invention can be used in the treatment of fungal infections of human and animal subjects (including but not limited to horses, cattle, sheep, dogs, cats, etc.) for medical and veterinary purposes. Examples of such infections include but are not limited to ailments such as Onychomycosis, sporotichosis, hoof rot, jungle rot, Pseudallescheria boydii, scopulariopsis or athletes foot, sometimes generally referred to as “white-line” disease, as well as fungal infections in immunocompromised patients such as AIDS patients and transplant patients. Thus, fungal infections may be of skin or of keratinaceous material such as hair, hooves, or nails, as well as systemic infections such as those caused by Candida spp., Cryptococcus neoformans, and Aspergillus spp., such as in pulmonary aspergillosis and Pneumocystis carinii pneumonia. Active compounds as described herein may be combined with a pharmaceutically acceptable carrier and administered or applied to such subjects or infections (e.g., topically, parenterally) in an amount effective to treat the infection in accordance with known techniques, as (for example) described in U.S. Pat. Nos. 6,680,073; 6,673,842; 6,664,292; 6,613,738; 6,423,519; 6,413,444; 6,403,063; and 6,042,845; the disclosures of which applicants specifically intend be incorporated by reference herein in their entirety.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically-acceptable carrier” as used herein means a pharmaceutically-acceptable material, composition or vehicle, such as a liquid or solid filler, diluent, excipient, solvent or encapsulating material, involved in carrying or transporting the subject peptidomimetic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the active ingredient which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a peptide or peptidomimetic of the present invention with liquid carriers, or finely divided solid carriers, or both, and then, if necessary, shaping the product.

The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Pharmaceutical compositions of this invention suitable for parenteral administration comprise one or more active compounds of the invention in combination with one or more pharmaccutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given by any suitable means of administration including orally, parenterally, topically, transdermally, rectally, etc. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Topical or parenteral administration is preferred.

“Parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Actual dosage levels of the active ingredients in the pharmaceutical compositions of this invention may be varied so as to obtain an amount of the active ingredient which is effective to achieve the desired therapeutic response, e.g., antimycotic activity, for a particular patient, composition, and mode of administration, without being toxic to the patient. The selected dosage level will depend upon a variety of factors including the activity of the particular active compound employed, the route of administration, the time of administration, the rate of excretion of the particular active compound being employed, the duration of the treatment, other drugs, compounds and/or materials used in combination with the particular inhibitor employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. As a general proposition, a dosage from about 0.01 or 0.1 to about 50, 100 or 200 mg/kg will have therapeutic efficacy, with all weights being calculated based upon the weight of the active compound, including the cases where a salt is employed.

The present invention is explained in greater detail in the following non-limiting Examples.

### EXAMPLE 1

### 2,4-Bis-(3-chlorophenyl)-3-[(3-pyridyl)hydroxymethyl]thiophene (Compound 1)

To a solution of 273 mg (2.0 mmol) of 3-chlorophenylacetylene in 4 mL of anhydrous THF under a N₂ atmosphere at −78° C. was added 1.25 mL (2.0 mmol) of a 1.6M solution of n-butyllithium in hexane. The solution was stirred for 1.5 hr, and then 64 mg (2.0 mmol) of sulfur was added. After an additional 1.5 hr at −78° C., the red solution was warmed to room temperature and added to a solution of 400 mg (1.66 mmol) of 3-(3-chlorophenyl)-1-(3-pyridyl)-2-propyn-1-one in 4 mL of THF and 1 mL of acetonitrile. The reaction solution was stirred for 2 hr at room temperature, and was then poured into water. The aqueous layer was extracted several times with ether. The combined ether extracts were washed with saturated sodium chloride and dried over magnesium sulfate. The drying agent was filtered off, and the ether was removed by rotoevaporation. The crude product was purified by flash column chromatography on silica gel to give 185 mg (0.45 mmol) of 2,4-bis-(3-chlorophenyl)-3-[(3-pyridyl)carbonyl]thiophene. ¹H NMR (CDCl₃): 7.95 (d of q, 1), 8.56 (d of d, 1), and 8.73 ppm (d, 1). MS m/z 410.0 (M+H).

To a solution of 32 mg (0.08 mmol) of 2,4-bis-(3-chlorophenyl)-3-[(3-pyridyl)carbonyl]thiophene in 2 mL of anhydrous THF was added 10 mg (0.26 mmol) of lithium aluminum hydride. The mixture was stirred at 0° C. for 0.5 hr and was then diluted with ethyl acetate. The ethyl acetate solution was washed with water and dried over magnesium sulfate. The drying agent was filtered off, and the solvent was removed by rotoevaporation. The crude product was purified by preparative thin layer chromatography (prep TLC) to give 30 mg (0.073 mmol) 2,4-bis-(3-chlorophenyl)-3-[(3-pyridyl)hydroxy-methyl]thiophene (Compound 1) in 91% yield. ¹H NMR (CDCl₃): 5.98 (br s, 1), 7.44 (br d, 1), 8.08 (br s, 1), and 8.21 ppm (br d, 1). MS m/z 412.0 (M+H).

### EXAMPLE 2

### 4-(4-Chlorophenyl)-2-(5-chloro-2-thienyl)-3-[(3-pyridyl)hydroxymethyl]thiophene (Compound 4)

To a solution of 137 mg (1.0 mmol) of 4-chlorophenylacetylene in 2 mL of anhydrous THF under a N₂ atmosphere at −78° C. was added 0.063 mL (1.0 mmol) of a 1.6M solution of n-butyllithium in hexane. The solution was stirred for 1.5 hr, and then 32 mg (1.0 mmol) of sulfur was added. After an additional 1.5 hr at −78° C., the red solution was warmed to −10° C. One half of the solution was added to a solution of 99 mg (0.39 mmol) of 3-(5-chloro-2-thienyl)-1-(3-pyridyl)-2-propyn-1-one in 2 mL of THF and 0.5 mL of acetonitrile. After an additional 0.5 hr, the reaction was diluted with ethyl acetate. The ethyl acetate solution was washed with saturated sodium chloride and dried over magnesium sulfate. The drying agent was filtered off, and the solvent was removed by rotoevaporation. The crude product was purified by flash column chromatography on silica gel to give 70 mg (0.17 mmol) of 4-(4-chlorophenyl)-2-(5-chloro-2-thienyl)-3-[(3-pyridyl)carbonyl]-thiophene. ¹H NMR (CDCl₃): 6.76 (d, 1), 6.95 (d, 1), 7.96 (br d, 1), 8.59 (br d, 1), and 8.73 ppm (br s, 1). MS m/z 415.9 (M+H).

To a solution of 70 mg (0.17 mmol) of 4-(4-chlorophenyl)-2-(5-chloro-2-thienyl)-3-[(3-pyridyl)carbonyl]thiophene in 3 mL of anhydrous THF was added 13 mg (0.34 mmol) of lithium aluminum hydride. The mixture was stirred at 0° C. for 0.5 hr and was then diluted with ethyl acetate and a minimum amount of water to decompose the LiAlH₄. The ethyl acetate solution was decanted off and evaporated to dryness. The crude product was purified by preparative thin layer chromatography (prep TLC) to give 60 mg (0.14 mmol) 4-(4-chlorophenyl)-2-(5-chloro-2-thienyl)-3-[(3-pyridyl)hydroxymethyl]thiophene (Compound 4) in 84% yield. ¹H NMR (CDCl₃): 6.08 (br s, 1), 6.81 (d, 1), 6.87 (d, 1), 7.39 (br d, 1), 8.18 (br s, 1), and 8.30 ppm (br d, 1). MS m/z 417.9 (M+H).

### EXAMPLE 3

### 3-(3-Chlorophenyl)-1-(3-pyridyl)-2-propyn-1-one

To a solution of 5.0 gm (36.6 mmol) of 3-chlorophenylacetylene in 30 mL of anhydrous THF under a N₂ atmosphere at −78° C. was added 23 mL (36.6 mmol) of a 1.6M solution of n-butyllithium in hexane. The solution was stirred for 2 hr, and then a solution of 3.9 gm (36.6 mmol) of pyridine-3-carboxaldehyde in 5 mL of THF was added. The reaction mixture was stirred at −78° C. for 2 hr and then was poured into ice water. The solution was extracted several times with ether. The combined ether extracts were washed twice with aqueous sodium bisulfite solution to remove any remaining aldehyde, then with water, and finally with saturated sodium chloride solution. The ether layer was dried over magnesium sulfate. The drying agent was filtered off, and the ether was removed by rotoevaporation to give 8.5 gm (34.7 mmol) of oily product, 3-(3-chlorophenyl)-1-(3-pyridyl)-2-propyn-1-ol.

The 8.5 gm of 3-(3-chlorophenyl)-1-(3-pyridyl)-2-propyn-1-ol in 50 mL of DMSO was added 10.7 gm (38 mmol) of o-iodosobenzoic acid (IBX) in portions. The resulting mixture was stirred for 2 hr at room temperature, and then was diluted with ethyl acetate and water. The solution was filtered and the filtrate was extracted with ethyl acetate. The combined ethyl acetate extracts were washed consecutively with water and saturated sodium chloride solution. The ethyl acetate layer was dried over magnesium sulfate, the drying agent was filtered off, and the solvent was removed by rotoevaporation to give 6.84 gm (28.3 mmol) of brown solid 3-(3-chlorophenyl)-1-(3-pyridyl)-2-propyn-1-one in an overall 77% crude yield. ¹H NMR (CDCl₃): 8.40 (d of m, 1), 8.84 (d of d, 1), and 9.40 ppm (d, 1). MS m/z 242.0 (M+H).

### EXAMPLE 4

### 2,4-Bis-(2,4-difluorophenyl)-3-[(3-pyridyl)hydroxymethyl]thiophene

To a suspension of 1.54 gm (11.1 mmol) of potassium carbonate, 1.44 gm (4.46 mmol) of tetrabutylammonium bromide, and 0.05 gm (0.22 mmol) of palladium(II) diacetate in 1.1 mL of acetonitrile/H₂O (9:1) under a N₂ atmosphere was added 1.83 gm (6.69 mmol) of 2,4-difluoro-1-iodobenzene and 0.50 gm (4.46 mmol) of thiophene-3-carboxaldehyde. The mixture was heated at 80° C. for 3 days, and then diluted with ethyl acetate. The ethyl acetate solution was washed with water and dried over magnesium sulfate. The drying agent was filtered off, and the solvent was removed by rotoevaporation to give a red-brown solid which was purified by flash column chromatography on silica gel to give a mixture of 2-(2,4-difluorophenyl)thiophene-3-carboxaldehyde and 2,4-bis-(2,4-difluorophenyl)thiophene-3-carboxaldehyde which was used in the next reaction.

To a solution of 0.41 gm (2.6 mmol) of 3-bromopyridine in 1.7 mL of anhydrous THF under a N₂ atmosphere was added 1.3 mL (2.6 mmol) of 2M i-propylmagnesium chloride in THF. After 2 hr of stirring, 0.39 gm of the above mixture of aldehydes in 2 mL of THF was added. After another 2 hrs, the reaction was diluted with water, and ethyl acetate was added to extract the products. The ethyl acetate extract was washed with saturated sodium chloride and dried over magnesium sulfate. The drying agent was filtered off, and the solvent was removed by rotoevaporation to give a mixture of products that were purified by preparative HPLC. From this reaction, 167 mg of 2-(2,4-difluorophenyl)-3-[(3-pyridyl)hydroxymethyl]thiophene and 96 mg of the desired 2,4-bis-(2,4-difluorophenyl)-3-[(3-pyridyl)hydroxymethyl]thiophene were isolated. For the latter, ¹H NMR (CDCl₃): 7.67 (br d of t, 1), 8.55 (d of d, 1), and 8.49 ppm (br d, 1). MS m/z 416.0 (M+H).

### EXAMPLE 5

### 2-(3-Chlorophenyl)-4,5-dimethyl-4-hydroxy-3-[(3-pyridyl)carbonyl]-4,5-dihydrothiophene

A solution of 0.20 gm (0.83 mmol) of 3-(3-chlorophenyl)-1-(3-pyridyl)-2-propyn-1-one, 0.10 gm (0.99 mmol) of 3-mercapto-2-butanone, and 0.072 mL (0.83 mmol) of morpholine in 3 mL of diethoxymethane was heated to reflux under a N₂ atmosphere for 8 hrs. The reaction mixture was diluted with ethyl acetate, and the organic solution was washed with saturated sodium chloride solution. The ethyl acetate layer was dried over magnesium sulfate, the drying agent was filtered off, and the solvent was removed by rotoevaporation. The crude product was purified by silica gel column chromatography to yield 0.18 gm (0.53 mmol) of 2-(3-chlorophenyl)-4,5-dimethyl-4-hydroxy-3-[(3-pyridyl)carbonyl]-4,5-dihydrothiophene as a mixture of two isomers. ¹H NMR (CDCl₃): 1.56 (d,3), 1.64 (s, 3), 3.80 (t, 1), 7.82 (d of m, 1), 8.45 (d of d, 1), and 8.64 ppm (d, 1). MS m/z 346.0 (M+H).

### EXAMPLE 6

### 2-(3-Chlorophenyl)-4,5-dimethyl-3-[(3-pyridyl)hydroxymethyl]thiophene (Compound 55)

A mixture of 0.050 gm (0.14 mmol) of 2-(3-chlorophenyl)-4,5-dimethyl-4-hydroxy-3-[(3-pyridyl)carbonyl]-4,5-dihydrothiophene as a mixture of two isomers and 0.024 mL of acetic anhydride in 1.0 mL of toluene was placed in a sealed vial and heated to 100° C. in a sand bath for 48 hrs. The crude reaction product was purified by preparative thin layer chromatography (prep TLC) to give 0.037 gm (0.11 mmol) of 2-(3-chlorophenyl)-4,5-dimethyl-3-[3-pyridylcarbonyl]thiophene. ¹H NMR (CDCl₃): 2.09 (s, 3), 2.43 (s, 3), 8.00 (d of m, 1), 8.58 (d of d, 1), and 8.78 ppm (d, 1). MS m/z 328.0 (M+H).

To a solution of 0.037 gm (0.11 mmol) of the preceding ketone, 2-(3-chlorophenyl)-4,5-dimethyl-3-[3-pyridylcarbonyl]thiophene, in 3 mL of diethyl ether was added 0.020 gm (0.45 mmol) of lithium aluminum hydride. The mixture was stirred at 0° C. for 0.5 hr and was then diluted with ethyl acetate and a minimum amount of water to decompose the LiAlH₄. The ethyl acetate solution was decanted off and evaporated to dryness. The crude product was purified by preparative thin layer chromatography (prep TLC) to give 0.032 gm (0.10 mmol) of 2-(3-chlorophenyl)-4,5-dimethyl-3-[(3-pyridyl)hydroxymethyl]thiophene (Compound 55). ¹H NMR (CDCl₃): 1.82 (s, 3), 2.31 (s, 3), 7.64 (d of m, 1), 8.41 (d of d, 1), and 8.46 ppm (br s, 1). MS m/z 330.0.0 (M+H).

### EXAMPLE 7

### Biological Screening

Fungicidal activity for the compounds described in this invention was determined using a microtiter plate format. In primary screening, test compounds in 1 μL of dimethylsulfoxide (DMSO) are delivered to individual wells of a 96-well microtiter plate. Then 100 μL of minimal media consisting of 1.5% agar is delivered to each well and allowed to cool. Finally, inoculation is carried out by the addition of 10 μL of an aqueous suspension of fungal spores to the surface of the solid agar. The plates are covered and incubated in a controlled environment at 20° C. Fungicidal activity is determined by visual inspection and photometric analysis of fungal growth after 3-5 days, depending on the pathogen. Commercial standards (azoxystrobin, benomyl, captan, chlorothalonil, famoxadone, flusilazole, and propiconazole) are included in all assays. Test pathogens include Septoria tritici, Stagonospora nodorum, Phytophthora infestans, Moniliniafructicola and Botrytis cinerea. Dose response data for compounds found to be fungicidal in primary screening are obtained by screening 3-fold serial dilutions of the test compound. Fungicidal activity, noted as IC50 values in μM concentration, for certain of the compounds covered in this invention is included in the following Table 1. The coefficient of variation (ratio of standard deviation to the mean) expressed in percentage is given in parentheses.

TABLE 1

| Compound | B. | | | | M. |
|---|---|---|---|---|---|
| Number | cinerea | P. infestans | S. nodorum | S. tritici | fructicola |
| 4 | B (c) | E | B | A (d) | A |
| 6 | B (d) | E | B (c) | A (b) | A |
| 8 | ND | E | B (d) | A (d) | ND |
| 9 | B (d) | E | A (d) | A (c) | A |
| 12 | A (d) | E | A (d) | A | A |
| 14 | B (d) | E | A (b) | A | A |

IC50(μM): A=≦0.1; B=0.11-1.0; C=1.1-10; D=11-100; E=>100; ND=Not determined
C.V. (%): (a)=0-5; (b)=6-15; (c)=16-30 (d)=>30

The foregoing is illustrative of the present invention, and is not to be construed as limiting thereof. The invention is defined by the following claims, with epqivalents of the claims to be included therein.

That which is claimed is:

1. A compound of formula I: 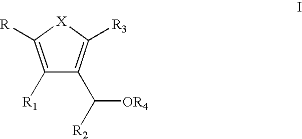
wherein:
X is S, O, or NR₅;
R is H; alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
R₁ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
R₂ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, or nitro; heteroaryl, especially 2-, 3- or 4-pyridyl optionally substituted with halogen, alkyl, alkenyl, alkynyl, haloalkyl, haloalkenyl, alkoxy, alkylthio, haloalkoxy, haloalkylthio, cyano, or nitro; 5-pyrimidinyl optionally substituted with halogen, alkyl, alkenyl, alkynyl, haloalkyl, haloalkenyl, alkoxy, alkylthio, haloalkoxy, haloalkylthio, cyano, or nitro; or 2- or 5-thiazolyl optionally substituted with halogen, alkyl, alkenyl, alkynyl, alkoxy, alkylthio, haloalkyl, haloalkenyl, haloalkoxy, haloalkylthio, cyano, or nitro;
R₃ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
R₄ is H; acyl; haloacyl; alkoxycarbonyl; aryloxycarbonyl; alkylaminocarbonyl; or dialkylaminocarbonyl;
R₅ is H; alkyl; alkenyl; alkynyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
or a salt thereof.

2. The compound of claim 1, wherein said compound is selected from the group consisting of compounds of formula Ia, compounds of formula Ib, and compounds of formula Ic: 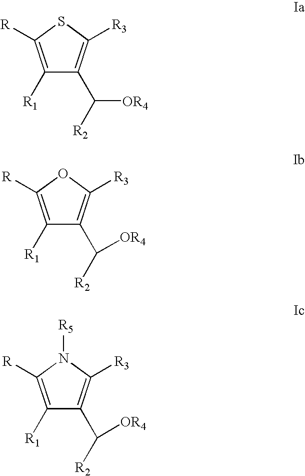
wherein R₁, R₂, R₃, R₄ and R₅ are as given above.

3. The compound of claim 1 wherein R is H or alkyl.

4. The compound of claim 1 wherein R₁ is aryl optionally substituted with halogen, alkyl, alkenyl, alkynyl, haloalkyl, haloalkenyl, alkoxy, alkylthio, haloalkoxy, haloalkylthio, cyano, nitro; or heteroaryl optionally substituted with halogen, alkyl, alkenyl, alkynyl, haloalkyl, haloalkenyl, alkoxy, alkylthio, haloalkoxy, haloalkylthio, cyano, or nitro.

5. The compound of claim 1 wherein R₁ is 2-chlorophenyl, 4-chlorophenyl, 2,4-dichlorophenyl, 2-fluorophenyl, 2,4-difluorophenyl, 3,5-difluorophenyl, 4-trifluoro-methylphenyl, 4-trifluoromethoxyphenyl, 2-thienyl, 3-thienyl, 5-chloro-2-thienyl, or 5-chloro-2-furyl.

6. The compound of claim 1 wherein R₁ is alkyl or arylalkyl optionally substituted with halogen, alkyl, alkenyl, alkynyl, haloalkyl, haloalkenyl, alkoxy, alkylthio, haloalkoxy, haloalkylthio, cyano, or nitro.

7. The compound of claim 1 wherein R₂ is heteroaryl optionally substituted with halogen, alkyl, alkenyl, alkynyl, haloalkyl, haloalkenyl, alkoxy, alkylthio, haloalkoxy, haloalkylthio, cyano, or nitro.

8. The compound of claim 1 wherein R₂ is 3-pyridyl or 5-pyrimidinyl optionally substituted with halogen, alkyl, alkenyl, alkynyl, haloalkyl, haloalkenyl, alkoxy, alkylthio, haloalkoxy, haloalkylthio, cyano, or nitro.

9. The compound of claim 1 wherein R₃ is alkyl; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, nitro; or alkylsilyl.

10. The compound of claim 1 wherein R₃ is phenyl, 3-chlorophenyl, 4-chlorophenyl, 4-fluorophenyl, 3,5-difluorophenyl, 4-methylphenyl, 2-thienyl, 5-chloro-2-thienyl, 5-methyl-2-thienyl, 3-thienyl, t-butyl, or trimethylsilyl.

11. The compound of claim 1 wherein R₄ is H.

12. The compound of claim 1 wherein:
R is H or alkyl;
R₁ is aryl optionally substituted with halogen, alkyl, alkenyl, alkynyl, haloalkyl, haloalkenyl, alkoxy, alkylthio, haloalkoxy, haloalkylthio, cyano, nitro; or heteroaryl optionally substituted with halogen, alkyl, alkenyl, alkynyl, haloalkyl, haloalkenyl, alkoxy, alkylthio, haloalkoxy, haloalkylthio, cyano, or nitro;
R₂ is heteroaryl optionally substituted with halogen, alkyl, alkenyl, alkynyl, haloalkyl, haloalkenyl, alkoxy, alkylthio, haloalkoxy, haloalkylthio, cyano, or nitro;
R₃ is alkyl; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, nitro; or alkylsilyl; and
R₄ is H;
R₅ is alkyl and haloalkyl;
or a salt thereof.

13. The compound of claim 12 wherein R₁ is 2-chlorophenyl, 4-chlorophenyl, 2,4-dichlorophenyl, 2-fluorophenyl, 2,4-difluorophenyl, 3,5-difluorophenyl, 4-trifluoro-methylphenyl, 4-trifluoromethoxyphenyl, 2-thienyl, 3-thienyl, 5-chloro-2-thienyl, or 5-chloro-2-furyl.

14. The compound of claim 12 wherein R₂ is 3-pyridyl or 5-pyrimidinyl optionally substituted with halogen, alkyl, alkenyl, alkynyl, haloalkyl, haloalkenyl, alkoxy, alkylthio, haloalkoxy, haloalkylthio, cyano, or nitro.

15. The compound of claim 12 wherein R₃ is phenyl, 3-chlorophenyl, 4-chlorophenyl, 4-fluorophenyl, 2,4-difluorophenyl, 3,5-difluorophenyl, 4-methylphenyl, 2-thienyl, 5-chloro-2-thienyl, 5-methyl-2-thienyl, 3-thienyl, t-butyl, or trimethylsilyl.

16. The compound of claim 12 selected from the group consisting of: 2,4-Bis-(3-chlorophenyl)-3-[(3-pyridyl)hydroxymethyl]thiophene (compound 1); 4-(3-Chlorophenyl)-2-(5-chloro-2-thienyl)-3-[(3-pyridyl)hydroxymethyl]thiophene (compound 2); 4-(3-Chlorophenyl)-2-(3,5-difluorophenyl)-3-[(3-pyridyl)hydroxymethyl]thiophene (compound 3); 4-(4-Chlorophenyl)-2-(5-chloro-2-thienyl)-3-[(3-pyridyl)hydroxymethyl]thiophene (compound 4); 4-(4-Chlorophenyl)-2-(3,5-difluorophenyl)-3-[(3-pyridyl)hydroxymethyl]thiophene (compound 5); 2-(4-Chlorophenyl)-4-(2,4-difluorophenyl)-3-[(3-pyridyl)hydroxymethyl]thiophene (compound 6); 4-(2,4-Difluorophenyl)-2-(1,1-dimethylethyl)-3-[(3-pyridyl)hydroxymethyl]thiophene(compound 7); 2,4-Bis-(4-chlorophenyl)-3-[(3-pyridyl)hydroxymethyl]thiophene (compound 8); 4-(4-Chlorophenyl)-3-[(3-pyridyl)hydroxymethyl]-2-(2-thienyl)thiophene (compound 9); 2-(4-Chlorophenyl)-4-(5-chloro-2-thienyl)-3-[(3-pyridyl)hydroxymethyl]thiophene (compound 10); 4-(5-Chloro-2-thienyl)-2-(2,4-difluorophenyl)-3-[(3-pyridyl)hydroxymethyl]thiophene (compound 11); 2-(4-Chlorophenyl)-3-[(3-pyridyl)hydroxymethyl]-4-(2-thienyl)thiophene (compound 12); 2-(2,4-Difluorophenyl)-3-[(3-pyridyl)hydroxymethyl]-4-(2-thienyl)thiophene (compound 13); 2-(2,4-Difluorophenyl)-3-[(3-pyridyl)hydroxymethyl]-4-(2-thienyl)thiophene (compound 14); 2-(4-Butylphenyl)-4-(5-methyl-2-thienyl)-3-[(3-pyridyl)hydroxymethyl]thiophene (compound 15); 2,4-Bis-(2,4-Difluorophenyl)-3-[(3-pyridyl)hydroxymethyl]thiophene (compound 16); 4-(4-Chlorophenyl)-2-(2,4-difluorophenyl)-3-[(3-pyridyl)hydroxymethyl]thiophene (compound 17); 2,4-Bis-(2-trifluoromethylphenyl)-3-[(3-pyridyl)hydroxymethyl]thiophene (compound 18); 2,4-Bis-(3-trifluoromethylphenyl)-3-[(3-pyridyl)hydroxymethyl]thiophene (compound 19); 2,4-Bis-(4-trifluoromethylphenyl)-3-[(3-pyridyl)hydroxymethyl]thiophene (compound 20); 4-(4-Chlorophenyl)-3-[(3-pyridyl)hydroxymethyl]-2-(3-thienyl)thiophene (compound 21); 2-(5-Bromo-2-thienyl)-4-(4-chlorophenyl)-3-[(3-pyridyl)hydroxymethyl]thiophene (compound 22); 4-(4-Chlorophenyl)-2-(5-methyl-2-thienyl)-3-[(3-pyridyl)hydroxymethyl]thiophene (compound 23); 2-(3,5-Difluorophenyl)-3-[(3-pyridyl)hydroxymethyl]-4-(3-thienyl)thiophene (compound 24); 2-(2,4-Difluorophenyl)-3-[(3-pyridyl)hydroxymethyl]-4-(3-thienyl)thiophene (compound 25); 2-(3,5-Difluorophenyl)-4-(4-fluorophenyl)-3-[(3-pyridyl)hydroxymethyl]thiophene (compound 26); 2-(2,4-Difluorophenyl)-4-(4-fluorophenyl)-3-[(3-pyridyl)hydroxymethyl]thiophene (compound 27); 2-(4-Chlorophenyl)-3-[(3-pyridyl)hydroxymethyl]-4-(3-thienyl)thiophene (compound 28); 3-[(3-Pyridyl)hydroxymethyl]-2-(2-tetrahydropyranyloxy-methyl)-4-(3-thienyl)thiophene (compound 29); 4-(2,4-Difluorophenyl)-3-[(3-pyridyl)hydroxymethyl]-2-(3-thienyl)thiophene (compound 32); 4-(2,4-Difluorophenyl)-3-[(3-pyridyl)hydroxymethyl]-2-(2-thienyl)thiophene (compound 39); 2-(2,4-Difluorophenyl)-4-(2-fluorophenyl)-3-[(3-pyridyl)hydroxymethyl]thiophene (compound 45); 2,4-Bis-(2-Chlorophenyl)-3-[(3-pyridyl)hydroxymethyl]-thiophene (compound 49); 2,4-Bis-(3-Chlorophenyl)-3-[(3-pyridyl)hydroxymethyl]thiophene (compound 50); 2,4-Bis-(Phenyl)-3-[(3-pyridyl)hydroxymethyl]thiophene (compound 51); 2,4-Bis-(2,4-Dichlorophenyl)-3-[(3-pyridyl)hydroxymethyl]thiophene (compound 52); 2,4-Bis-(2-Fluorophenyl)-3-[(3-pyridyl)hydroxymethyl]thiophene (compound 53); 2,4-Bis-(3-Fluorophenyl)-3-[(3-pyridyl)hydroxymnethyl]thiophene (compound 54); 2-(3-Chlorophenyl)-4,5-dimethyl-3-[(3-pyridyl)hydroxymethyl]thiophene (compound 55); 4-(5-Chloro-2-furanyl)-2-(4-chlorophenyl)-3-[(3-pyridyl)hydroxymethyl]thiophene (compound 56); 4-(5-Chloro-2-furanyl)-2-(2,4-difluorophenyl)-3-[(3-pyridyl)hydroxymethyl]thiophene (compound 57); 2,4-Bis-(2-thienyl)-3-[(3-pyridyl)hydroxymethyl]thiophene (compound 58); 2,4-Bis-(4-Fluorophenyl)-3-[(3-pyridyl)hydroxymethyl]thiophene (compound 59); and 2-(3-Chlorophenyl)-4-phenyl-3-[(3-pyridyl)hydroxymethyl]thiophene (compound 60); 2,4-Bis-(3-chloro-5-trifluoromethylphenyl)-3-[(3-pyridyl)hydroxymethyl]-thiophene (compound 61); 2,4-Bis-(2,5-difluorophenyl)-3-[(3-pyridyl)hydroxymethyl]thiophene (compound 62); 2,4-Bis-(4-chloro-3-fluorophenyl)-3-[(3-pyridyl)hydroxymethyl]thiophene (compound 63); 2,4-Bis-(3-Methoxyphenyl)-3-[(3-pyridyl)hydroxymethyl]-thiophene (compound 64); 4-(2-Fluorophenyl)-3-[(3-pyridyl)hydroxymethyl]-2-(2-thienyl)thiophene (compound 65); 2,4-Bis-(2-chloro-4-trifluoromethylphenyl)-3-[(3-pyridyl)hydroxymethyl]thiophene (compound 66); 2,4-Bis-(4-Methoxyphenyl)-3-[(3-pyridyl)hydroxymethyl]thiophene (compound 67); 2-(3-Chlorophenyl)-4-(2,4-difluorophenyl)-3-[(3-pyridyl)hydroxymethyl]-thiophene (compound 68); 2-(5-Bromo-2-thienyl)-4-(2,4-difluorophenyl)-3-[(3-pyridyl)hydroxymethyl]thiophene (compound 69); 2-(5-Chloro-2-thienyl)-4-(2,4-difluorophenyl)-3-[(3-pyridyl)hydroxymethyl]thiophene (compound 70); 5-Chloro-2-(5-chloro-2-thienyl)-4-(2,4-difluorophenyl)-3-[(3-pyridyl)hydroxymethyl]-thiophene (compound 71); 4-(4-Chlorophenyl)-2-(2-fluorophenyl)-3-[(3-pyridyl)hydroxymethyl]thiophene (compound 72); 4-(4-Chlorophenyl)-2-(3-fluorophenyl)-3-[(3-pyridyl)hydroxymethyl]thiophene (compound 73); 2-(2-Chlorophenyl)-4-(2,4-difluorophenyl)-3-[(3-pyridyl)hydroxymethyl]thiophene (compound 74); 4-(2,4-Difluorophenyl)-2-(2-fluorophenyl)-3-[(3-pyridyl)hydroxymethyl]thiophene (compound 75); 2-(4-Chlorophenyl)-4-(4-chloro-2-fluorophenyl)-3-[(3-pyridyl)hydroxymethyl]thiophene (compound 76); 2-(3-Chlorophenyl)-4-(4-chloro-2-fluorophenyl)-3-[(3-pyridyl)hydroxymethyl]-thiophene (compound 77); 4-(2,4-Difluorophenyl)-2-(4-fluorophenyl)-3-[(3-pyridyl)hydroxymethyl]thiophene (compound 78); and salts thereof.

17. A composition for controlling and preventing plant pathogenic microorganisms comprising, in combination, a compound of claim 1 together with a suitable carrier.

18. The composition of claim 17, further comprising at least one additional fungicide or SAR inducer.

19. A method of controlling or preventing infestation of cultivated plants by pathogenic microorganisms, comprising:
applying a compound according to claim 1 to said plants, parts thereof or the locus thereof in an amount effective to control said microorganisms.

20. A method according to claim 19, wherein the microorganism is a fungal organism.

21. The method of claim 20, wherein said fungal organism is selected from the group consisting of Septoria tritici, Stagonospora nodoruin, Phytophthora infestans, Botrytis cinerea, and Monilinia fructicola.

22. A method of controlling or preventing infestation of plant propagation material by pathogenic microorganisms, comprising:
applying a compound according to claim 1 to said plant propagation material in an amount effective to control said microorganisms.

23. The method of claim 22, wherein said plant propagation material comprises seeds.

24. A method according to claim 22, wherein the microorganism is a fungal organism.

25. A method of controlling or preventing infestation of a technical material by pathogenic microorganisms, comprising:
applying a compound according to claim 1 to said technical material in an amount effective to control said microorganisms.

26. A method of treating a fungal infection in a subject in need thereof, comprising:
administering a compound of claim 1 or a pharmaceutically acceptable salt thereof to said subject in an amount effective to treat said fungal infection.

27. A composition for treating a fungal infection in a subject in need thereof, comprising, in combination, a compound of claim 1 or a pharmaceutically acceptable salt thereof together with a pharmaceutically acceptable carrier.

28. A method of making a compound of formula Ia: 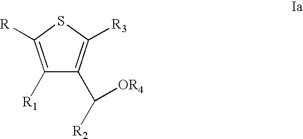
wherein:
R is H;
R₁ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
R₂ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, or nitro; heteroaryl, especially 2-, 3- or 4-pyridyl optionally substituted with halogen, alkyl, alkenyl, alkynyl, haloalkyl, haloalkenyl, alkoxy, alkylthio, haloalkoxy, haloalkylthio, cyano, or nitro; 5-pyrimidinyl optionally substituted with halogen, alkyl, alkenyl, alkynyl, haloalkyl, haloalkenyl, alkoxy, alkylthio, haloalkoxy, haloalkylthio, cyano, or nitro; or 2- or 5-thiazolyl optionally substituted with halogen, alkyl, alkenyl, alkynyl, alkoxy, alkylthio, haloalkyl, haloalkenyl, haloalkoxy, haloalkylthio, cyano, or nitro;
R₃ is alkyl; alkoxyalkyl; haloalkyl; arylalkyl optionally substituted with halogen, alkyl, alkenyl, alkynyl, haloalkyl, haloalkenyl, alkoxy, alkylthio, haloalkoxy, haloalkylthio, cyano, or nitro; aryloxyalkyl optionally substituted with halogen, alkyl, alkenyl, alkynyl, haloalkyl, haloalkenyl, alkoxy, alkylthio, haloalkoxy, haloalkylthio, cyano, or nitro; arylthioalkyl optionally substituted with halogen, alkyl, alkenyl, alkynyl, haloalkyl, haloalkenyl, alkoxy, alkylthio, haloalkoxy, haloalkylthio, cyano, or nitro; aryl optionally substituted with halogen, alkyl, alkenyl, alkynyl, haloalkyl, haloalkenyl, alkoxy, alkylthio, haloalkoxy, haloalkylthio, cyano, nitro; heteroaryl optionally substituted with halogen, alkyl, alkenyl, alkynyl, haloalkyl, haloalkenyl, alkoxy, alkylthio, haloalkoxy, haloalkylthio, cyano, or nitro; or alkylsilyl;
R₄ is H; acyl (e.g., acetyl, benzoyl, phenylacetyl); haloacyl; alkoxycarbonyl; aryloxycarbonyl; alkylaminocarbonyl; or dialkylaminocarbonyl;
said method comprising:
(a) reacting an acetylenethiolate of formula II 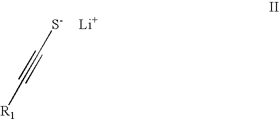
where R₁ is as given above with an acetylenic ketone of formula III: 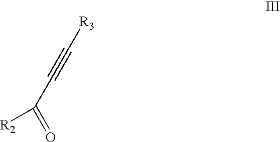
where R₂ and R₃ are as given above in an inert solvent to produce a compound of formula IV, 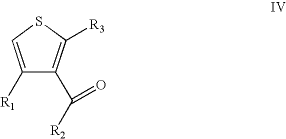
and then:
(b) reducing said compound of Formula IV to produce said compound of Formula Ia
(R=H).

29. The method of claim 28, wherein said reducing step is carried out with LiAlH₄ in an inert solvent or with NaBH₄ in an alcoholic solvent.
